(19) United States
(12) Patent Application Publication
(10) Pub. No.: US 2007/0207984 A1
(43) Pub. Date: Sep. 6, 2007

(54) Cyclohexylamino Benzene, Pyridine, and Pyridazine Derivatives

(76) Inventors: Kenneth He Huang, Chapel Hill, NC (US); Jeron Eaves, Chapel Hill, NC (US); James Veal, Apex, NC (US); Steven E. Hall, Chapel Hill, NC (US); Thomas E. Barta, Carrboro, NC (US); Gunnar J. Hanson, Dallas, TX (US)

Correspondence Address: MCDONNELL BOEHNEN HULBERT & BERGHOFF LLP, 300 S. WACKER DRIVE, 32ND FLOOR, CHICAGO, IL, 60606, US

(21) Appl. No.: 11/678,975

(22) Filed: Feb. 26, 2007

Related U.S. Application Data
(60) Provisional application No. 60/777,124, filed on Feb. 27, 2006.

(51) Int. Cl. A61K 31/675; C07D 231/56; C07D 209/02; A61K 31/405

(52) U.S. Cl. 514/80; 514/406; 514/415; 548/113; 548/361.1; 514/414; 548/465; 548/469

(57) ABSTRACT

Disclosed are compounds and pharmaceutically acceptable salts of Formula (I), 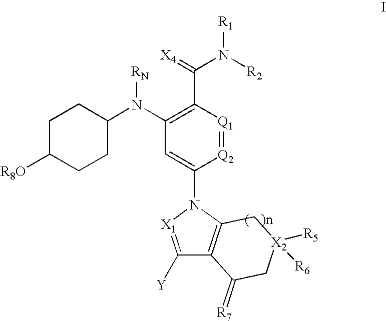
wherein Q₁, Q₂, R_{N}, R₁, R₂, R₅, R₆, R₇, R₈, X₁, X₂, X₄, and Y are as defined herein. Compounds of Formula (I) are useful in the treatment of diseases and/or conditions related to cell proliferation, such as cancer, inflammation, arthritis, angiogenesis, or the like. Also disclosed are pharmaceutical compositions comprising compounds of the invention and methods of treating the aforementioned conditions using such compounds.

This application claims priority of U.S. application Ser. No. 60/777,124, filed Feb. 27, 2006, the disclosure of which is incorporated herein in its entirety.

### BACKGROUND OF THE INVENTION

1. Field of the Invention

The invention relates to benzene, pyridine, and pyridazine derivatives and more specifically to such compounds that are useful in the treatment and/or prevention of diseases and/or conditions related to cell proliferation, such as cancer, inflammation and inflammation-associated disorders, and conditions associated with angiogenesis.

2. Description of the Related Art

Cancer is characterized by abnormal cellular proliferation. Cancer cells exhibit a number of properties that make them dangerous to the host, typically including an ability to invade other tissues and to induce capillary ingrowth, which assures that the proliferating cancer cells have an adequate supply of blood. A hallmark of cancerous cells is their abnormal response to control mechanisms that regulate cell division in normal cells and continue to divide until they ultimately kill the host.

Angiogenesis is a highly regulated process under normal conditions, however many diseases are driven by persistent unregulated angiogenesis. Unregulated angiogenesis may either cause a particular disease directly or exacerbate an existing pathological condition. For example, ocular neovascularization has not only been implicated as the most common cause of blindness, but also is believed the dominant cause of many eye diseases. Further, in certain existing conditions, for example arthritis, newly formed capillary blood vessels invade the joints and destroy cartilage, or in the case of diabetes, new capillaries formed in the retina invade the vitreous, bleed, and cause blindness. Growth and metastasis of solid tumors are also dependent on angiogenesis (Folkman, J., Cancer Res., 46, 467-473 1986; and Folkman, J., Journal of the National Cancer Institute, 82, 4-6 1989). It has been shown, for example, that tumors which enlarge to greater than 2 mm must obtain their own blood supply and do so by inducing the growth of new capillary blood vessels. Once these new blood vessels become embedded in the tumor, they provide a means for tumor cells to enter the circulation and metastasize to distant sites such as liver, lung or bone (Weidner, N., et al. The New England Journal of Medicine, 324(1), 1-8 1991). Under conditions of unregulated angiogenesis, therapeutic methods designed to control, repress, and/or inhibit angiogenesis could lead to the abrogation or mitigation of these conditions and diseases.

Inflammation is related to a variety of disorders such as pain, headaches, fever, arthritis, asthma, bronchitis, menstrual cramps, tendonitis, bursitis, psoriasis, eczema, burns, dermatitis, inflammatory bowel syndrome, Crohn's disease, gastritis, irritable bowel syndrome, ulcerative colitis, vascular diseases, Hodgkin's disease, sclerodoma, rheumatic fever, type I diabetes, myasthenia gravis, sarcoidosis, nephrotic syndrome, Behcet's syndrome, polymyositis, hypersensitivity, conjunctivitis, gingivitis, post-injury swelling, myocardial ischemia, and the like.

Heat-shock protein 90 (HSP-90) is a cellular chaperone protein required for the activation of several eukaryotic protein kinases, including the cyclin-dependent kinase CDK4. Geldanamycin, an inhibitor of the protein-refolding activity of HSP-90, has been shown to have antiproliferative and antitumor activities.

HSP-90 also guides the intracellular disposition and proteolytic turnover of many key regulators of cell growth and survival. Its function is subverted during oncogenesis to make malignant transformation possible and to facilitate rapid somatic evolution, and to allow mutant proteins to retain or even gain function. Inhibition of HSP-90 slows these processes and thus has therapeutic use (Whitesell L, Lindquist, S. L., Nature Rev. Cancer, 2005, 10, 761-72).

Ansamycin antibiotics, e.g., herbimycin A (HA), geldanamycin (GM), and 17-allylaminogeldanamycin (17-AAG) are thought to exert their anticancerous effects by tight binding of the N-terminus pocket of HSP-90, thereby destabilizing substrates that normally interact with HSP-90 (Stebbins, C. et al. Cell 1997, 89, 239-250). This pocket is highly conserved and has weak homology to the ATP-binding site of DNA gyrase (Stebbins, C. et al., supra; Grenert, J. P. et al. J. Biol. Chem. 1997, 272, 23843-50).

In vitro and in vivo studies have demonstrated that occupancy of this N-terminal pocket by ansamycins and other HSP-90 inhibitors alters HSP-90 function and inhibits protein folding. At high concentrations, ansamycins and other HSP-90 inhibitors have been shown to prevent binding of protein substrates to HSP-90 (Scheibel, T. H. et al. Proc. Natl. Acad. Sci. USA 1999, 96, 1297-302; Schulte, T. W. et al. J. Biol. Chem. 1995, 270, 24585-8; Whitesell, L. et al. Proc. Natl.Acad. Sci. USA 1994, 91, 8324-8328). Ansamycins have also been demonstrated to inhibit the ATP-dependent release of chaperone-associated protein substrates (Schneider, C. L. et al. Proc. Natl. Acad. Sci., USA 1996, 93, 14536-41; Sepp-Lorenzinoet et al. J. Biol Chem. 1995, 270, 16580-16587). In either event, the substrates are degraded by a ubiquitin-dependent process in the proteasome (Schneider, C. L., supra Sepp-Lorenzino, L., et al. J. Biol. Chem. 1995, 270, 16580-16587; Whitesell, L. et al. Proc. Natl. Acad. Sci. USA 1994, 91, 8324-8328). HSP-90 substrate destabilization occurs in tumor and non-transformed cells alike and has been shown to be especially effective on a subset of signaling regulators, e.g., Raf (Schulte, T. W. et al., Biochem. Biophys. Res. Commun. 1997, 239, 655-9 Schulte, T. W., et al., J. Biol. Chem. 1995, 270, 24585-8), nuclear steroid receptors (Segnitz, B.; U. Gehring J. Biol. Chem. 1997, 272, 18694-18701; Smith, D. F. et al. Mol. Cell Biol. 1995, 15, 6804-12), v-Src (Whitesell, L. et al. Proc. Natl. Acad. Sci. USA 1994, 91, 8324-8328) and certain transmembrane tyrosine kinases (Sepp-Lorenzino, L. et al. J. Biol. Chem. 1995, 270, 16580-16587) such as EGF receptor (EGFR) and HER2/Neu (Hartmann, F., et al. Int. J. Cancer 1997,70, 221-9; Miller, P. et al. Cancer Res. 1994,54, 2724-2730; Mimnaugh, E. G., et al. J. Biol. Clzem. 1996,271, 22796-801 ; Schnur, R. et al. J. Med. Chenu. 1995, 38, 3806-3812), CDK4, and mutant p53 (Erlichman et al. Proc. AACR 2001, 42, abstract 4474). The ansamycin-induced loss of these proteins leads to the selective disruption of certain regulatory pathways and results in growth arrest at specific phases of the cell cycle (Muise-Heimericks, R. C. et al. J. Biol. Chem. 1998, 273, 29864-72), and apoptosis, and/or differentiation of cells so treated (Vasilevskaya, A. et al. Cancer Res., 1999, 59, 3935-40). Therefore, compounds of the invention, as inhibitors of HSP-90, are useful for the treatment and/or prevention of many types of cancers and proliferative disorders, and are also useful in combination therapies with radiation treatments or other chemotherapy agents.

Inhibition of HSP-90 is also known to result in up regulation of the expression of the chaperone HSP-70. HSP-70 up regulation is considered to be of therapeutic benefit for treatment of a wide range of neurodegenerative diseases including, but not limited to: Alzheimer's disease; Parkinson's disease; Dementia with Lewy bodies; Amyotropic lateral scleriosis (ALS); Polyglutamine disease; Huntington's disease; Spinal and bulbar muscular atrophy (SBMA); and Spinocerebellar ataxias (SCA1-3,7). Therefore, compounds of the invention are useful for treatment of such neurodegenerative diseases (Muchowski, P. J., Wacker J. L., Nat. Rev. Neurosci. 2005, 6, 11-22; Shen H. Y., et al. J. Biol. Chem. 2005, 280, 39962-9).

Inhibition of HSP-90 also has anti-fungal activity, both as a stand alone therapy and in combination with standard anti-fungal therapies such as the azole class of drugs. Therefore, the compounds of the invention are useful for treatment of fungal infections including, but not limited to, systemic fungal infections (Cowen, L. E., Lindquist, S., Science 2005, 309, 2185-9; and Cell. 1997 Apr 18;89(2):239-50).

Inhibition of HSP-90 of Plasmodium falciparum, a protozoan parasite that causes malaria in humans, produces antimalarial activity. Therefore, compounds of the invention, as inhibitors of this protein, are useful as antimalarial drugs (Rajinder Kumar, Alla Musiyenko, Sailen Barik Malaria Journal 2003 2:30)

Therefore, there is a continuing need in the art for new methods of treating cancer, inflammation and inflammation-associated disorders, neurodegenerative diseases, fungal infections, malaria, and conditions or diseases related to uncontrolled angiogenesis.

### SUMMARY OF THE INVENTION

The invention encompasses compounds of Formula (I), pharmaceutical compositions containing compounds of Formula (I), and methods for the treatment of diseases and/or conditions related to inflammation, arthritis, angiogenesis, neurodegenerative disease, fungal infections, malaria, and cell proliferation such as cancer.

In one aspect, the invention provides compounds of Formula (I), 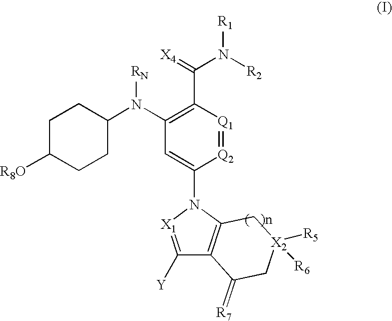
or a pharmaceutically acceptable salt thereof, wherein
Q₁ and Q₂ are independently N or CR_{Q}, wherein
   each R_{Q} is independently hydrogen, halogen, cyano, —C(O)OH, —C(O)—O(C₁-C₆ alkyl), —N(R_{N})₂, C₁-C₆ alkyl, C₁-C₆ haloalkyl, C₃-C₇ cycloalkyl, aryl, or heteroaryl, wherein
      each alkyl, cycloalkyl, aryl, and heteroaryl group is optionally substituted with from 1-4 groups that are independently C₁-C₆ alkyl, C₁-C₆ alkoxy, halogen, hydroxy, amino, mono- or di-(C₁-C₆)alkylamino, halo(C₁-C₆)alkyl, halo(C₁-C₆)alkoxy, or carboxamide;
each R_{N} is independently —H, —C₁-C₆ alkyl-, —C₁-C₆ haloalkyl, or —C(O)Z, wherein
   Z is —C₁-C₆ alkyl, —C₁-C₆ haloalkyl, -aryl, —OR′, wherein
      R′ is —C₁-C₆ alkyl, —C₁-C₆ haloalkyl, cycloalkyl, aryl, heterocycloalkyl, or heteroaryl; wherein
         each alkyl, cycloalkyl, aryl, heterocycloalkyl, and heteroaryl group is optionally substituted with from 1-4 groups that are independently C₁-C₆ alkyl, C₁-C₆ alkoxy, halogen, hydroxy, amino, mono- or di-(C₁-C₆)alkylamino, halo(C₁-C₆)alkyl, halo(C₁-C₆)alkoxy, or carboxamide;
R₁ and R₂ are independently H or C₁-C₆ alkyl;
R₇ is O, S, NH, N—OH, N—NH₂, N—NHR′, or N—O—R′;
R₈ is —P(O)(OR_{P})₂, —S(O)₂OR_{S}, or -T-V—NH(R₂₀);
each R_{P} is —H, —C₁-C₁₀ alkyl, —C₁-C₁₀ haloalkyl, or aryl, wherein
   the C₁-C₁₀ alkyl or aryl is optionally substituted with one
   or more groups independently selected from C₁-C₆ alkyl,
   C₁-C₆ alkoxy, halogen, hydroxy, amino, mono- or di-(C₁-C₆)alkylamino, nitro, halo(C₁-C₆)alkyl, carboxy, or carboxamide;
R_{S} is —H, —C₁-C₁₀ alkyl, —C₁-C₁₀ haloalkyl, or aryl, wherein the C₁-C₁₀ alkyl or aryl is optionally substituted with one or more groups indpendently selected from C₁-C₆ alkyl, C₁-C₆ alkoxy, halogen, cyano, hydroxy, amino, mono- or di-(C₁-C₆)alkylamino, nitro, halo(C₁-C₆)alkyl, carboxy, or carboxamide;
T is —C(O)—, —C(O)O—, —C(O)N(R_{N})—, —S(O)—, or —S(O)₂;
V is C₂-C₁₀ alkyl optionally substituted with at least one group
   selected from C₁-C₆ alkyl, C₁-C₆ alkoxy, halogen, hydroxy, amino, mono- or di-(C₁-C₆)alkylamino, nitro, halo(C₁-C₆)alkyl, carboxy, carboxamide, or —NH—C(O)—C₁-C₆ alkoxy; or
V is V₂-L-V₁;
V₁ is —C₁-C₁₀ alkyl-, —C₃-C₇ cycloalkyl-, -aryl-, or -heteroaryl-;
L is —O—, —C(O)—, —OC(O), —C(O)O—, —N(R_{N})C(O)—, —C(O)N(R_{N})—, —OC(O)N(R_{N})—, —N(R_{N})C(O)O—, or —N(R_{N})C(O)N(R_{N})—;
V₂ is —C₁-C₁₀ alkyl-, —C₃-C₇ cycloalkyl-, -aryl-, or -heteroaryl-;
   wherein each V₁ and V₂ independently are optionally substituted with at least one group selected from C₁-C₆ alkyl, C₁-C₆ alkoxy, halogen, hydroxy, amino, mono- or
   di-(C₁-C₆)alkylamino, nitro, halo(C₁-C₆)alkyl, carboxy, carboxamide, or —NH—C(O)—C₁-C₆ alkoxy;
R₂₀ is —H or —C(O)—C₁-C₆ alkoxy;
X₁ represents N or CR_{X} wherein R_{X} is —H or C₁-C₆ alkyl;
X₂ is C or N;
X₄ is O, S, NH, N—OH, N—NH₂, N—NHaryl, N—NH—(C₁-C₆ alkyl), or N—O—R′;
Y is hydrogen, halogen, C₁-C₁₀ alkyl, C₁-C₁₀ haloalkyl, C₃-C₇ cycloalkyl, C₃-C₇ cycloalkyl(C₁-C₁₀)alkyl, C₁-C₆ acyl, aryl or heteroaryl, wherein
   each C₁-C₁₀ alkyl or C₃-C₇ cycloalkyl group is optionally substituted with aryl or heteroaryl, wherein
      the aryl and heteroaryl groups are optionally substituted with 1-4 groups that are independently C₁-C₆ alkyl, C₁-C₆ alkoxy, halogen, hydroxy, amino, mono- or di-(C₁-C₆)alkylamino, nitro, halo(C₁-C₆)alkyl, halo(C₁-C₆)alkoxy, or carboxamide; and
n is 0, 1, 2, 3, or 4,
provided that when
   (i) X₂ is C, then
      R₅ and R₆ are independently H or C₁-C₆ alkyl, or
      R₅ and R₆ together with the carbon to which they are attached form a 5-8 membered ring; and
   (ii) X₂ is N, then
      R₆ is absent and R₅ is H or C₁-C₆ alkyl.

In another aspect, the invention provides intermediates that are useful in making the compounds of the invention.

In another aspect, the present invention provides pharmaceutical compositions comprising a compound of Formula (I), or a pharmaceutically acceptable salt thereof, and at least one pharmaceutically acceptable carrier, solvent, adjuvant or diluent.

In another aspect, the present invention provides a method of treating inflammation, arthritis, angiogenesis, neurodegenerative disease, fungal infections, malaria, or disorders related to cell proliferation such as cancer comprising administering a therapeutically effective amount of a compound of Formula (I) or a pharmaceutically acceptable salt thereof to a patient in need of such treatment.

In another aspect, the invention provides a method of treating inflammation, arthritis, angiogenesis, neurodegenerative disease, fungal infections, malaria, or disorders related to cell proliferation such as cancer comprising administering a pharmaceutical composition to a patient in need of such treatment, wherein the pharmaceutical composition comprises a therapeutically effective amount of a compound of Formula (I), or a pharmaceutically acceptable salt thereof, and at least one pharmaceutically acceptable carrier, solvent, adjuvant or diluent.

In another aspect, the invention provides the use of a compound of Formula (I), or a pharmaceutically acceptable salt thereof, for the manufacture of a medicament for treating inflammation, arthritis, angiogenesis, neurodegenerative disease, fungal infections, malaria, or disorders related to cell proliferation such as cancer.

In another aspect, the invention provides methods of preparing the compounds of the invention and the intermediates used in those methods.

In another aspect, the present invention comprises a method of treating a disease or condition related to cell proliferation comprising administering a therapeutically effective amount of a compound of Formula (I) or a pharmaceutically acceptable salt thereof to a patient in need of such treatment.

In another aspect, the present invention provides method of treating inflammation or arthritis comprising administering a therapeutically effective amount of a compound of Formula (I) or a pharmaceutically acceptable salt thereof to a patient in need of such treatment.

In another aspect, the invention provides method of treating a disease or disorder related to the activity of heat shock protein 90 and its respective client proteins, comprising administering a therapeutically effective amount to a patient in need of such treatment a compound of Formula (I) or a pharmaceutically acceptable salt thereof.

In another aspect, the invention provides a method of treating a disease or disorder related to the activity of heat shock protein 90, comprising administering to a patient in need of such treatment a therapeutically effective amount of a compound of Formula (I) or a pharmaceutically acceptable salt thereof, wherein the HSP-90 mediated disorder is selected from the group consisting of inflammatory diseases, fungal infections, autoimmune disorders, stroke, ischemia, cardiac disorders, neurological disorders, fibrogenetic disorders, proliferative disorders, tumors, leukemias, neoplasms, cancers, carcinomas, metabolic diseases and malignant disease.

In another aspect, the invention provides a method of treating fibrogenetic disorder comprising administering to a patient in need of such treatment a therapeutically effective amount of a compound of Formula (I) or a pharmaceutically acceptable salt thereof, wherein the fibrogenetic disorder is selected from the group consisting of scleroderma, polymyositis, systemic lupus, rheumatoid arthritis, liver cirrhosis, keloid formation, interstitial nephritis and pulmonary fibrosis.

In another aspect, the invention provides a method of protecting a patient from infection caused by an organism selected from Plasmodium species, preferably Plasmodium falciparum, comprising administering a therapeutically effective amount of a compound of Formula (I) or a pharmaceutically acceptable salt thereof, to the patient at risk of infection due to exposure to such organism.

In another aspect, the invention provides a method of reducing the level of infection in a patient where the infection is caused by an organism selected from Plasmodium species, preferably Plasmodium falciparum comprising administering to an infected patient a therapeutically effective amount of a compound of Formula (I) or a pharmaceutically acceptable salt thereof.

In another aspect, the invention provides a method of treating a metazoan parasite infection comprising administering a therapeutically effective amount of a compound of Formula (I) or a pharmaceutically acceptable salt thereof to a patient in need of such treatment.

In another aspect, the invention comprises a method of treating a metazoan parasite infection wherein the parasite is Plasmodium falciparum comprising administering a therapeutically effective amount of a compound of Formula (I) or a pharmaceutically acceptable salt thereof to a patient in need of such treatment.

In another aspect, the invention provides a kit comprising a compound of Formula (I), or a pharmaceutically acceptable salt thereof, and instructions for using the compound or salt of Formula (I).

In another aspect, the invention provides a kit comprising a pharmaceutical composition and instructions for using the pharmaceutical composition wherein the pharmaceutical composition comprises a compound of Formula (I), or a pharmaceutically acceptable salt thereof, and at least one pharmaceutically acceptable carrier, solvent, adjuvant or diluent.

### DETAILED DESCRIPTION OF THE INVENTION

In another aspect, the invention provides compounds of Formula (II) 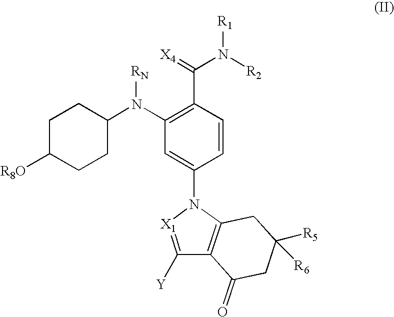
or a pharmaceutically acceptable salt thereof wherein R_{N}, R₁, R₂, R₅, R₆, R₈, X₁, X₄, and Y are as defined in Formula (I).

In another aspect, the invention provides compounds of Formula (II) wherein R₅ and R₆ are independently H or C₁-C₆ alkyl; and R_{N}, R₁, R₂, R₈, X₁, X₄, and Y are as defined in Formula (I).

In another aspect, the invention provides a compound of Formula (II), wherein R₅ and R₆ are independently H or C₁-C₆ alkyl; X₄ is O; and R_{N}, R₁, R₂, R₈, X₁, and Y are as defined in Formula (I).

In another aspect, the invention provides a compound of Formula (II), wherein R₅ and R₆ are independently C₁-C₆ alkyl, and R_{N}, R₁, R₂, R₈, X₁, X₄, and Y are as defined in Formula (I).

In another aspect, the invention provides a compound of Formula (III), 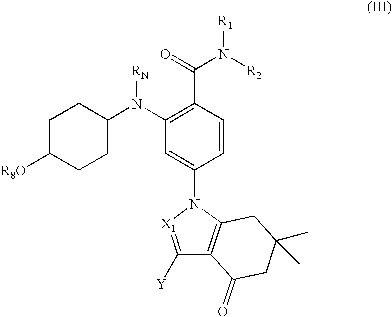
or a pharmaceutically acceptable salt thereof, wherein R_{N}, R₁, R₂, R₈, X₁, and Y are as defined in Formula (I).

In another aspect, the invention provides a compound of Formula (III), wherein R_{N} is —H or —C₁-C₁₀ alkyl, and R₁, R₂, R₈, X₁, and Y are as defined in Formula (I).

In another aspect, the invention provides a compound of Formula (III), wherein X₁ is N, CH or C(CH₃), and R_{N}, R₁, R₂, R₈, and Y are as defined in Formula (I).

In another aspect, the invention provides a compound of Formula (IV), 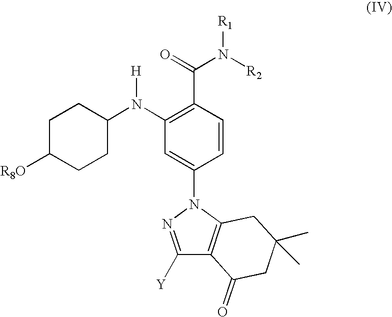
or a pharmaceutically acceptable salt thereof, wherein R₁, R₂, R₈, and Y are as defined in Formula (I).

In another aspect, the invention provides a compound of Formula (IV), wherein Y is hydrogen, halogen, C₁-C₁₀ alkyl, C₃-C₇ cycloalkyl(C₁-C₁₀)alkyl, or C₁-C₁₀ haloalkyl; and R₁, R₂, and R₈ are as defined in Formula (I).

In another aspect, the invention provides a compound of Formula (IV), wherein Y is C₁-C₁₀ alkyl, C₁-C₁₀ haloalkyl, or C₃-C₇ cycloalkyl(C₁-C₁₀)alkyl; R₈ is —P(O)(OR_{P})₂; R_{P} is —H or —C₁-C₁₀ alkyl, and R₁ and R₂ are independently H or C₁-C₆ alkyl.

In another aspect, the invention provides a compound of Formula (IV), wherein Y is methyl, ethyl, trifluoromethyl, or cyclopropylmethyl; R₈ is —P(O)(OR_{P})₂; R_{P} is —H or —C₁-C₁₀ alkyl, and R₁ and R₂ are independently H or C₁-C₆ alkyl.

In another aspect, the invention provides a compound of Formula (IV), wherein Y is C₁-C₁₀ alkyl, C₁-C₁₀ haloalkyl, or C₃-C₇ cycloalkyl(C₁-C₁₀)alkyl; R₈ is —S(O)₂OR_{S}; R_{S} is —H or —C₁-C₁₀ alkyl; and R₁ and R₂ are independently H or C₁-C₆ alkyl.

In another aspect, the invention provides a compound of Formula (IV), wherein Y is methyl, ethyl, trifluoromethyl, or cyclopropylmethyl; R₈ is —S(O)₂OR_{S}; R_{S} is —H or —C₁-C₁₀ alkyl; and R₁ and R₂ are independently H or C₁-C₆ alkyl.

In another aspect, the invention provides a compound of Formula (IV), wherein R₈ is -T-V—NH(R₂₀); T is —C(O)—, —C(O)O—, —C(O)N(R_{N})—, —S(O)—, or —S(O)₂; and V is C₂-C₁₀ alkyl optionally substituted with at least one group selected from C₁-C₆ alkyl, C₁-C₆ alkoxy, halogen, hydroxy, amino, mono- or di-(C₁-C₆)alkylamino, nitro, halo(C₁-C₆)alkyl, carboxy, carboxamide, or —NH—C(O)—C₁-C₆ alkoxy; or V is V₂-L-V₁; V₁ is —C₁-C₁₀ alkyl-, —C₃-C₇ cycloalkyl-, -aryl-, or -heteroaryl-; L is —O—, —C(O)—, —OC(O), —C(O)O—, —N(R_{N})C(O)—, —C(O)N(R_{N})—, —OC(O)N(R_{N})—, —N(R_{N})C(O)O—, or —N(R_{N})C(O)N(R_{N})—; V₂ is —C₁-C₁₀ alkyl-, —C₃-C₇ cycloalkyl-, -aryl-, or -heteroaryl-; wherein each V₁ and V₂ independently is optionally substituted with at least one group which is C₁-C₆ alkyl, C₁-C₆ alkoxy, halogen, hydroxy, amino, mono- or di-(C₁-C₆)alkylamino, nitro, halo(C₁-C₆)alkyl, carboxy, or carboxamide; R₂₀ is —H or —C(O)—C₁-C₆ alkoxy; and R₁, R₂, and Y are as defined in Formula (I).

In another aspect, the invention provides a compound of Formula (IV) wherein the 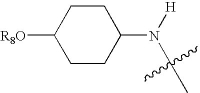
group is trans; Y is C₁-C₁₀ alkyl, C₁-C₁₀ haloalkyl, or C₃-C₇ cycloalkyl(C₁-C₁₀)alkyl; R₁ and R₂ are independently H or C₁-C₆ alkyl; R₈ is —P(O)(OR_{P})₂; each R_{P} is independently —H or —C₁-C₁₀ alkyl.

In another aspect, the invention provides a compound of Formula (IV) wherein the 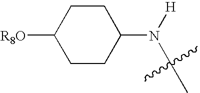
group is trans; Y is methyl, ethyl, trifluoromethyl, or cyclopropylmethyl; R₁ and R₂ are H; R₈ is —P(O)(OR_{P})₂, each R_{P} is —H; R_{S} is —H.

In another aspect, the invention provides a compound of Formula (IV) wherein the 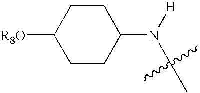
group is trans; Y is C₁-C₁₀ alkyl, C₁-C₁₀ haloalkyl, or C₃-C₇ cycloalkyl(C₁-C₁₀)alkyl; R₁ and R₂ are independently H or C₁-C₆ alkyl; R₈ is —S(O)₂OR_{S}; and R_{S} is —H or —C₁-C₁₀ alkyl.

In another aspect, the invention provides a compound of Formula (IV) wherein the 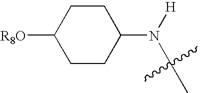
group is trans; Y is methyl, ethyl, trifluoromethyl, or cyclopropylmethyl; R₁ and R₂ are H; R₈ is —S(O)₂OR_{S}; and R_{S} is —H or —C₁-C₁₀ alkyl;

In another aspect, the invention provides a compound of Formula (IV) wherein the 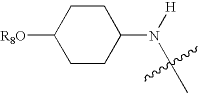
group is trans; Y is C₁-C₁₀ alkyl, C₁-C₁₀ haloalkyl, or C₃-C₇ cycloalkyl(C₁-C₁₀)alkyl; R₁ and R₂ are independently H or C₁-C₆ alkyl; R₈ is -T-V—NH(R₂₀); T is —C(O)—; V is —C₂-C₁₀ alkyl- optionally substituted with 1 amino or —NH—C(O)—C₁-C₆ alkoxy group; or V is V₁—N(R_{N})C(O)—V₂; V₁ is —C₁-C₁₀ alkyl- optionally substituted with 1 amino or —NH—C(O)—C₁-C₆ alkoxy group; V₂ is —C₁-C₁₀ alkyl- optionally substituted with 1 amino or —NH—C(O)—C₁-C₆ alkoxy group; R_{N} is —H or —C₁-C₆ alkyl-; and R₂₀ is —H or —C(O)—C₁-C₆ alkoxy.

In another aspect, the invention provides a compound of Formula (IV) wherein the 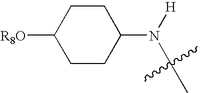
group is trans; Y is methyl, ethyl, trifluoromethyl, or cyclopropylmethyl; R₁ and R₂ are H; R₈ is -T-V—NH(R₂₀); T is —C(O)—; V is —C₂-C₁₀ alkyl- optionally substituted with 1 amino or —NH—C(O)—C₁-C₆ alkoxy group; and R₂₀ is —H or —C(O)—C₁-C₆ alkoxy.

In another aspect, the invention provides a compound of Formula (IV) wherein the 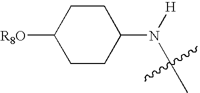
group is trans; Y is methyl, ethyl, trifluoromethyl, or cyclopropylmethyl; R₁ and R₂ are H; R₈ is -T-V—NH(R₂₀); T is —C(O)—; V is V₁—N(R_{N})C(O)—V₂; V₁ is —C₁-C₁₀ alkyl- optionally substituted with 1 amino or —NH—C(O)—C₁-C₆ alkoxy group; V₂ is —C₁-C₁₀ alkyl- optionally substituted with 1 amino or —NH—C(O)—C₁-C₆ alkoxy group; R_{N} is —H; and R₂₀ is —H or —C(O)—C₁-C₆ alkoxy.

In another aspect, the invention provides a method of treating inflammation comprising administering a therapeutically effective amount of a compound of Formula (IV), or a pharmaceutically acceptable salt thereof, to a patient in need of such treatment.

In another aspect, the invention provides a method of treating arthritis comprising administering a therapeutically effective amount of a compound of Formula (IV), or a pharmaceutically acceptable salt thereof, to a patient in need of such treatment.

In another aspect, the invention provides a method of treating angiogenesis comprising administering a therapeutically effective amount of a compound of Formula (IV), or a pharmaceutically acceptable salt thereof, to a patient in need of such treatment.

In another aspect, the invention provides a method of treating neurodegenerative disease comprising administering a therapeutically effective amount of a compound of Formula (IV), or a pharmaceutically acceptable salt thereof, to a patient in need of such treatment.

In another aspect, the invention provides a method of treating fungal infections comprising administering a therapeutically effective amount of a compound of Formula (IV), or a pharmaceutically acceptable salt thereof, to a patient in need of such treatment.

In another aspect, the invention provides a method of treating malaria comprising administering a therapeutically effective amount of a compound of Formula (IV), or a pharmaceutically acceptable salt thereof, to a patient in need of such treatment.

In another aspect, the invention provides a method of treating a disease or disorder related to cell proliferation comprising administering a therapeutically effective amount of a compound of Formula (IV), or a pharmaceutically acceptable salt thereof, to a patient in need of such treatment.

In another aspect, the invention provides a method of inhibiting cell proliferation comprising administering a therapeutically effective amount of a compound of Formula (IV), or a pharmaceutically acceptable salt thereof, to a patient in need of such treatment.

In another aspect, the invention provides a method of treating a disease or disorder related to Heat-shock protein 90 comprising administering a therapeutically effective amount of a compound of Formula (IV), or a pharmaceutically acceptable salt thereof, to a patient in need of such treatment.

In another aspect, the invention provides a method of inhibiting Heat-shock protein 90 comprising administering a therapeutically effective amount of a compound of Formula (IV), or a pharmaceutically acceptable salt thereof, to a patient in need of such treatment.

In another aspect, the invention provides a method of treating cancer comprising administering a therapeutically effective amount of a compound of Formula (IV), or a pharmaceutically acceptable salt thereof, to a patient in need of such treatment.

In another aspect, the invention provides a pharmaceutical composition comprising a therapeutically effective amount of a compound of Formula (IV), or a pharmaceutically acceptable salt thereof, and at least one pharmaceutically acceptable carrier, solvent, adjuvant or diluent.

In another aspect, the invention provides a method of treating Plasmodium falciparum comprising administering a therapeutically effective amount of a compound of Formula (IV) or a pharmaceutically acceptable salt thereof to a patient in need of such treatment.

In another aspect, the invention comprises a compound of Formula (V) 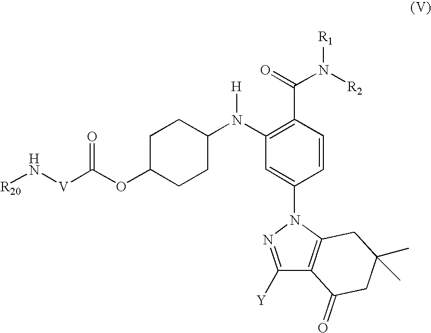
or a pharmaceutically acceptable salt thereof wherein R₁, R₂, R₂₀, and V are as defined in Formula (I).

In another aspect, the invention comprises a compound of Formula (V wherein Y is hydrogen, halogen, C₁-C₁₀ alkyl, C₃-C₇ cycloalkyl(C₁-C₁₀)alkyl, or C₁-C₁₀ haloalkyl, and R₁, R₂, R₂₀, and V are as defined in Formula (I).

In another aspect, the invention provides a compound of Formula (V), wherein Y is hydrogen, halogen, C₁-C₁₀ alkyl, C₃-C₇ cycloalkyl(C₁-C₁₀)alkyl, or C₁-C₁₀ haloalkyl; V is —C₂-C₁₀ alkyl- optionally substituted with at least one group selected from C₁-C₆ alkyl, C₁-C₆ alkoxy, halogen, hydroxy, amino, mono- or di-(C₁-C₆)alkylamino, nitro, halo(C₁-C₆)alkyl, carboxy, carboxamide, or —NH—C(O)—C₁-C₆ alkoxy; or V is V₂-L-V₁; V₁ is —C₂-C₁₀ alkyl- optionally substituted with at least one group selected from C₁-C₆ alkyl, C₁-C₆ alkoxy, halogen, hydroxy, amino, mono- or di-(C₁-C₆)alkylamino, nitro, halo(C₁-C₆)alkyl, carboxy, carboxamide, or —NH—C(O)—C₁-C₆ alkoxy; L is —O—, —OC(O), —C(O)O—, —N(R_{N})C(O)—, or —C(O)N(R_{N})—; V₂ is —C₂-C₁₀ alkyl- optionally substituted with at least one group selected from C₁-C₆ alkyl, C₁-C₆ alkoxy, halogen, hydroxy, amino, mono- or di-(C₁-C₆)alkylamino, nitro, halo(C₁-C₆)alkyl, carboxy, carboxamide, or —NH—C(O)—C₁-C₆ alkoxy; and R₁ R₂, and R₂₀ are as defined in Formula (I).

In another aspect, the invention provides a compound of Formula (V), wherein Y is hydrogen, halogen, C₁-C₁₀ alkyl, C₃-C₇ cycloalkyl(C₁-C₁₀)alkyl, or C₁-C₁₀ haloalkyl; V is —C₂-C₁₀ alkyl- optionally substituted with at least one group selected from C₁-C₆ alkyl, C₁-C₆ alkoxy, halogen, hydroxy, amino, mono- or di-(C₁-C₆)alkylamino, nitro, halo(C₁-C₆)alkyl, carboxy, carboxamide, or —NH—C(O)—C₁-C₆ alkoxy; or V is V₂-L-V₁; V₁ is —C₂-C₁₀ alkyl- optionally substituted with at least one group selected from C₁-C₆ alkyl, C₁-C₆ alkoxy, halogen, hydroxy, amino, mono- or di-(C₁-C₆)alkylamino, nitro, halo(C₁-C₆)alkyl, carboxy, carboxamide, or —NH—C(O)—C₁-C₆ alkoxy; L is —N(R_{N})C(O)—; V₂ is —C₂-C₁₀ alkyl- optionally substituted with at least one group selected from C₁-C₆ alkyl, C₁-C₆ alkoxy, halogen, hydroxy, amino, mono- or di-(C₁-C₆)alkylamino, nitro, halo(C₁-C₆)alkyl, carboxy, carboxamide, or —NH—C(O)—C₁-C₆ alkoxy; and R₁, R₂, and R₂₀ are as defined in Formula (IV).

In another aspect, the invention provides a compound of Formula (V), wherein Y is hydrogen, halogen, C₁-C₁₀ alkyl, C₃-C₇ cycloalkyl(C₁-C₁₀)alkyl, or C₁-C₁₀ haloalkyl; V is —C₂-C₁₀ alkyl-; or V is V₂-L-V₁; V₁ is -C₁-C₁₀ alkyl-; L is —N(H)C(O)—; V₂ is —C₁-C₁₀ alkyl-; R₁ and R₂ are each —H; and R₂₀ is —H or —C(O)—C₁-C₆ alkoxy.

In another aspect, the invention provides a compound of Formula (V) wherein the 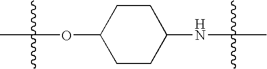
group is trans; Y is C₁-C₁₀ alkyl, C₁-C₁₀ haloalkyl, or C₃-C₇ cycloalkyl(C₁-C₁₀)alkyl; R₁ and R₂ are independently H or C₁-C₆ alkyl; V is —C₂-C₁₀ alkyl- optionally substituted with 1 amino or —NH—C(O)—C₁-C₆ alkoxy group; or V is V₁—N(R_{N})C(O)—V₂; V₁ is —C₁-C₁₀ alkyl- optionally substituted with 1 amino or —NH—C(O)—C₁-C₆ alkoxy group; V₂ is —C₁-C₁₀ alkyl- optionally substituted with 1 amino or —NH—C(O)—C₁-C₆ alkoxy group; R_{N} is —H or C₁-C₆ alkyl; and R₂₀ is —H or —C(O)—C₁-C₆ alkoxy.

In another aspect, the invention provides a compound of Formula (V) wherein the 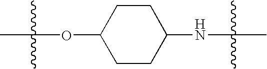
group is trans; Y is methyl, ethyl, trifluoromethyl, or cyclopropylmethyl; R₁ and R₂ are H; V is —C₂-C₁₀alkyl- optionally substituted with 1 amino or —NH—C(O)—C₁-C₆ alkoxy group; and R₂₀ is —H or —C(O)—C₁-C₆ alkoxy.

In another aspect, the invention provides a compound of Formula (V) wherein the 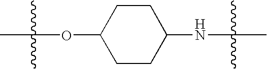
group is trans; Y is methyl, ethyl, trifluoromethyl, or cyclopropylmethyl; R₁ and R₂ are H; V is V₁—N(R_{N})C(O)—V₂; V₁ is —C₁-C₁₀ alkyl- optionally substituted with 1 amino or —NH—C(O)—C₁-C₆ alkoxy group; V₂ is —C₁-C₁₀ alkyl- optionally substituted with 1 amino or —NH—C(O)—C₁-C₆alkoxy group; R_{N} is —H; and R₂₀ is —H or —C(O)—C₁-C₆ alkoxy.

In another aspect, the invention provides a method of treating inflammation comprising administering a therapeutically effective amount of a compound of Formula (V), or a pharmaceutically acceptable salt thereof, to a patient in need of such treatment.

In another aspect, the invention provides a method of treating arthritis comprising administering a therapeutically effective amount of a compound of Formula (V), or a pharmaceutically acceptable salt thereof, to a patient in need of such treatment.

In another aspect, the invention provides a method of treating angiogenesis comprising administering a therapeutically effective amount of a compound of Formula (V), or a pharmaceutically acceptable salt thereof, to a patient in need of such treatment.

In another aspect, the invention provides a method of treating neurodegenerative disease comprising administering a therapeutically effective amount of a compound of Formula (V), or a pharmaceutically acceptable salt thereof, to a patient in need of such treatment.

In another aspect, the invention provides a method of treating fungal infections comprising administering a therapeutically effective amount of a compound of Formula (V), or a pharmaceutically acceptable salt thereof, to a patient in need of such treatment.

In another aspect, the invention provides a method of treating malaria comprising administering a therapeutically effective amount of a compound of Formula (V), or a pharmaceutically acceptable salt thereof, to a patient in need of such treatment.

In another aspect, the invention provides a method of treating a disease or disorder related to cell proliferation comprising administering a therapeutically effective amount of a compound of Formula (V), or a pharmaceutically acceptable salt thereof, to a patient in need of such treatment.

In another aspect, the invention provides a method of inhibiting cell proliferation comprising administering a therapeutically effective amount of a compound of Formula (V), or a pharmaceutically acceptable salt thereof, to a patient in need of such treatment.

In another aspect, the invention provides a method of treating a disease or disorder related to Heat-shock protein 90 comprising administering a therapeutically effective amount of a compound of Formula (V), or a pharmaceutically acceptable salt thereof, to a patient in need of such treatment.

In another aspect, the invention provides a method of inhibiting Heat-shock protein 90 comprising administering a therapeutically effective amount of a compound of Formula (V), or a pharmaceutically acceptable salt thereof, to a patient in need of such treatment.

In another aspect, the invention provides a method of treating cancer comprising administering a therapeutically effective amount of a compound of Formula (V), or a pharmaceutically acceptable salt thereof, to a patient in need of such treatment.

In another aspect, the invention provides a pharmaceutical composition comprising a therapeutically effective amount of a compound of Formula (V), or a pharmaceutically acceptable salt thereof, and at least one pharmaceutically acceptable carrier, solvent, adjuvant or diluent.

In another aspect, the invention provides a method of treating Plasmodium falciparum comprising administering a therapeutically effective amount of a compound of Formula (V) or a pharmaceutically acceptable salt thereof to a patient in need of such treatment.

In another aspect, the invention provides a compound of Formula (VI), 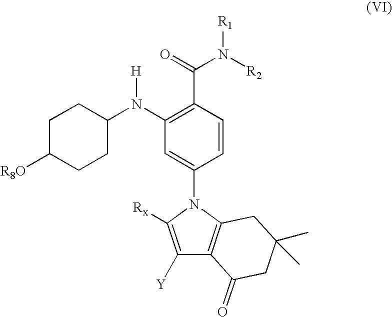
or a pharmaceutically acceptable salt thereof, wherein R₁, R₂, R₈, R_{X} and Y are as defined in Formula (I).

In another aspect, the invention provides a compound of Formula (VI), wherein R_{X} is H or CH₃; Y is hydrogen, halogen, C₁-C₁₀ alkyl, C₃-C₇ cycloalkyl(C₁-C₁₀)alkyl, or C₁-C₁₀ haloalkyl, and R₁, R₂, and R₈ are as defined in Formula (I).

In another aspect, the invention provides a compound of Formula (VI), wherein Y is hydrogen, halogen, C₁-C₁₀ alkyl, C₃-C₇ cycloalkyl(C₁-C₁₀)alkyl, or C₁-C₁₀ haloalkyl; R₈ is -T-V—NH(R₂₀); T is —C(O)—, —C(O)O—, —C(O)N(R_{N})—, —S(O)—, or —S(O)₂; V is C₂-C₁₀ alkyl optionally substituted with at least one group selected from C₁-C₆ alkyl, C₁-C₆ alkoxy, halogen, hydroxy, amino, mono- or di-(C₁-C₆)alkylamino, nitro, halo(C₁-C₆)alkyl, carboxy, carboxamide, or —NH—C(O)—C₁-C₆ alkoxy; or V is V₂-L-V₁; V₁ is —C₁-C₁₀ alkyl-, —C₃-C₇ cycloalkyl-, -aryl-, or -heteroaryl-; L is —O—, —C(O)—, —OC(O), —C(O)O—, —N(R_{N})C(O)—, —C(O)N(R_{N})—, —OC(O)N(R_{N})—, —N(R_{N})C(O)O—, or —N(R_{N})C(O)N(R_{N})—; V₂ is —C₁-C₁₀ alkyl-, —C₃-C₇ cycloalkyl-, -aryl-, or -heteroaryl-; each V₁ and V₂ independently are optionally substituted with at least one group selected from C₁-C₆ alkyl, C₁-C₆ alkoxy, halogen, hydroxy, amino, mono- or di-(C₁-C₆) alkylamino, nitro, halo(C₁-C₆)alkyl, carboxy, carboxamide, or —NH—C(O)—C₁-C₆ alkoxy; R₂₀ is —H or —C(O)—C₁-C₆ alkoxy; and R_{X}, R₁, and R₂ are as defined in Formula (I).

In another aspect, the invention provides a compound of Formula (VI), wherein R_{X} is H or CH₃; Y is C₁-C₁₀ alkyl, C₁-C₁₀ haloalkyl, or C₃-C₇ cycloalkyl(C₁-C₁₀)alkyl; R₈ is —P(O)(OR_{P})₂; R_{P} is —H or —C₁-C₁₀ alkyl, and R₁ and R₂ are independently H or C₁-C₆ alkyl.

In another aspect, the invention provides a compound of Formula (VI), wherein R_{X} is H or CH₃; Y is methyl, ethyl, trifluoromethyl, or cyclopropylmethyl; R₈ is —P(O)(OR_{P})₂; R_{P} is —H or —C₁-C₁₀ alkyl, and R₁ and R₂ are independently H or C₁-C₆ alkyl.

In another aspect, the invention provides a compound of Formula (VI), wherein R_{X} is H or CH₃; Y is C₁-C₁₀ alkyl, C₁-C₁₀ haloalkyl, or C₃-C₇ cycloalkyl(C₁-C₁₀)alkyl; R₈ is —S(O)₂OR_{S}; R_{S} is —H or —C₁-C₁₀ alkyl; and R₁ and R₂ are independently H or C₁-C₆ alkyl.

In another aspect, the invention provides a compound of Formula (VI), wherein R_{X} is H or CH₃; Y is methyl, ethyl, trifluoromethyl, or cyclopropylmethyl; R₈ is —S(O)₂OR_{S}; R_{S} is —H or —C₁-C₁₀ alkyl; and R₁ and R₂ are independently H or C₁-C₆ alkyl.

In another aspect, the invention provides a compound of Formula (VI) wherein the 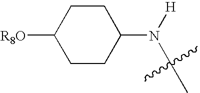
group is trans; R_{X} is H or CH₃; Y is C₁-C₁₀ alkyl, C₁-C₁₀ haloalkyl, or C₃-C₇ cycloalkyl(C₁-C₁₀)alkyl; R₁ and R₂ are independently H or C₁-C₆ alkyl; R₈ is —P(O)(OR_{P})₂; each R_{P} is independently —H or —C₁-C₁₀ alkyl.

In another aspect, the invention provides a compound of Formula (VI) wherein the 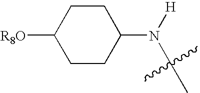
group is trans; R_{X} is H or CH₃; Y is methyl, ethyl, trifluoromethyl, or cyclopropylmethyl; R₁ and R₂ are H; R₈ is —P(O)(OR_{P})₂, each R_{P} is —H; R₈ is —H.

In another aspect, the invention provides a compound of Formula (VI) wherein the 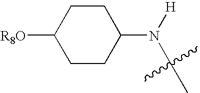
group is trans; R_{X} is H or CH₃; Y is C₁-C₁₀ alkyl, C₁-C₁₀ haloalkyl, or C₃-C₇ cycloalkyl(C₁-C₁₀)alkyl; R₁ and R₂ are independently H or C₁-C₆ alkyl; R₈ is —S(O)₂OR_{S}; and R_{S} is —H or —C₁-C₁₀ alkyl.

In another aspect, the invention provides a compound of Formula (VI) wherein the 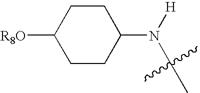
group is trans; R_{X} is H or CH₃; Y is methyl, ethyl, trifluoromethyl, or cyclopropylmethyl; R₁ and R₂ are H; R₈ is —S(O)₂OR_{S}; and R_{S} is —H or —C₁-C₁₀ alkyl;

In another aspect, the invention provides a compound of Formula (VI) wherein the 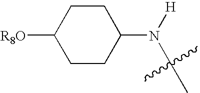
group is trans; R_{X} is H or CH₃; Y is C₁-C₁₀ alkyl, C₁-C₁₀ haloalkyl, or C₃-C₇ cycloalkyl(C₁-C₁₀)alkyl; R₁ and R₂ are independently H or C₁-C₆ alkyl; R₈ is -T-V—NH(R₂₀); T is —C(O)—; V is —C₂-C₁₀ alkyl- optionally substituted with 1 amino or —NH—C(O)—C₁-C₆ alkoxy group; or V is V₁—N(R_{N})C(O)—V₂; V₁ is —C₁-C₁₀ alkyl- optionally substituted with 1 amino or —NH—C(O)—C₁-C₆ alkoxy group; V₂ is —C₁-C₁₀ alkyl- optionally substituted with 1 amino or —NH—C(O)—C₁-C₆ alkoxy group; R_{N} is —H or —C₁-C₆ alkyl-; and R₂₀ is —H or —C(O)—C₁-C₆ alkoxy.

In another aspect, the invention provides a compound of Formula (IV) wherein the 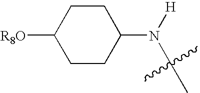
group is trans; R_{X} is H or CH₃; Y is methyl, ethyl, trifluoromethyl, or cyclopropylmethyl; R₁ and R₂ are H; R₈ is -T-V—NH(R₂₀); T is —C(O)—; V is —C₂-C₁₀ alkyl- optionally substituted with 1 amino or —NH—C(O)—C₁-C₆ alkoxy group; and R₂₀ is —H or —C(O)—C₁-C₆ alkoxy.

In another aspect, the invention provides a compound of Formula (IV) wherein the 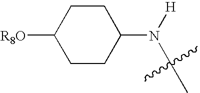
group is trans; R_{X} is H or CH₃; Y is methyl, ethyl, trifluoromethyl, or cyclopropylmethyl; R₁ and R₂ are H; R₈ is -T-V—NH(R₂₀); T is —C(O)—; V is V₁—N(R_{N})C(O)—V₂; V₁ is —C₁-C₁₀ alkyl- optionally substituted with 1 amino or —NH—C(O)—C₁-C₆ alkoxy group; V₂ is —C₁-C₁₀ alkyl- optionally substituted with 1 amino or —NH—C(O)—C₁-C₆ alkoxy group; R_{N} is —H; and R₂₀ is —H or —C(O)—C₁-C₆ alkoxy.

In another aspect, the invention provides a method of treating inflammation comprising administering a therapeutically effective amount of a compound of Formula (VI), or a pharmaceutically acceptable salt thereof, to a patient in need of such treatment.

In another aspect, the invention provides a method of treating arthritis comprising administering a therapeutically effective amount of a compound of Formula (VI), or a pharmaceutically acceptable salt thereof, to a patient in need of such treatment.

In another aspect, the invention provides a method of treating angiogenesis comprising administering a therapeutically effective amount of a compound of Formula (VI), or a pharmaceutically acceptable salt thereof, to a patient in need of such treatment.

In another aspect, the invention provides a method of treating neurodegenerative disease comprising administering a therapeutically effective amount of a compound of Formula (VI), or a pharmaceutically acceptable salt thereof, to a patient in need of such treatment.

In another aspect, the invention provides a method of treating fungal infections comprising administering a therapeutically effective amount of a compound of Formula (VI), or a pharmaceutically acceptable salt thereof, to a patient in need of such treatment.

In another aspect, the invention provides a method of treating malaria comprising administering a therapeutically effective amount of a compound of Formula (VI), or a pharmaceutically acceptable salt thereof, to a patient in need of such treatment.

In another aspect, the invention provides a method of treating a disease or disorder related to cell proliferation comprising administering a therapeutically effective amount of a compound of Formula (VI), or a pharmaceutically acceptable salt thereof, to a patient in need of such treatment.

In another aspect, the invention provides a method of inhibiting cell proliferation comprising administering a therapeutically effective amount of a compound of Formula (VI), or a pharmaceutically acceptable salt thereof, to a patient in need of such treatment.

In another aspect, the invention provides a method of treating a disease or disorder related to Heat-shock protein 90 comprising administering a therapeutically effective amount of a compound of Formula (VI), or a pharmaceutically acceptable salt thereof, to a patient in need of such treatment.

In another aspect, the invention provides a method of inhibiting Heat-shock protein 90 comprising administering a therapeutically effective amount of a compound of Formula (VI), or a pharmaceutically acceptable salt thereof, to a patient in need of such treatment.

In another aspect, the invention provides a method of treating cancer comprising administering a therapeutically effective amount of a compound of Formula (VI), or a pharmaceutically acceptable salt thereof, to a patient in need of such treatment.

In another aspect, the invention provides a pharmaceutical composition comprising a therapeutically effective amount of a compound of Formula (VI), or a pharmaceutically acceptable salt thereof, and at least one pharmaceutically acceptable carrier, solvent, adjuvant or diluent.

In another aspect, the invention provides a method of treating Plasmodium falciparum comprising administering a therapeutically effective amount of a compound of Formula (VI) or a pharmaceutically acceptable salt thereof to a patient in need of such treatment.

In another aspect, the invention provides a compound of Formula (VII) 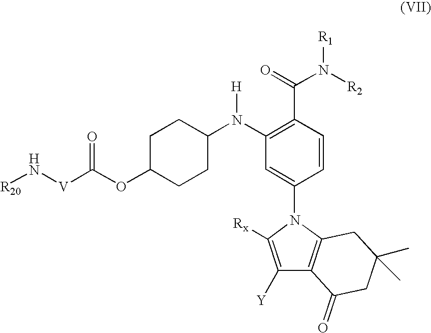
or a pharmaceutically acceptable salt thereof, wherein R_{X}, Y, R₁, R₂, R₂₀, and V are as defined in Formula (I).

In another aspect, the invention provides a compound of Formula (VII) wherein R_{X} is H or CH₃; Y is hydrogen, halogen, C₁-C₁₀ alkyl, C₃-C₇ cycloalkyl(C₁-C₁₀)alkyl, or C₁-C₁₀ haloalkyl, and R₁, R₂, R₂₀, and V are as defined in Formula (I).

In another aspect, the invention provides a compound of Formula (VII), wherein R_{X} is H or CH₃; Y is hydrogen, halogen, C₁-C₁₀ alkyl, C₃-C₇ cycloalkyl(C₁-C₁₀)alkyl, or C₁-C₁₀ haloalkyl; V is —C₂-C₁₀ alkyl- optionally substituted with at least one group selected from C₁-C₆ alkyl, C₁-C₆ alkoxy, halogen, hydroxy, amino, mono- or di-(C₁-C₆)alkylamino, nitro, halo(C₁-C₆)alkyl, carboxy, carboxamide, or —NH—C(O)—C₁-C₆ alkoxy; or V is V₂-L-V₁; V₁ is —C₂-C₁₀ alkyl- optionally substituted with at least one group selected from C₁-C₆ alkyl, C₁-C₆ alkoxy, halogen, hydroxy, amino, mono- or di-(C₁-C₆)alkylamino, nitro, halo(C₁-C₆)alkyl, carboxy, carboxamide, or —NH—C(O)—C₁-C₆ alkoxy; L is —O—, —OC(O), —C(O)O—, —N(R_{N})C(O)—, or —C(O)N(R_{N})—; V₂ is —C₂-C₁₀ alkyl- optionally substituted with at least one group selected from C₁-C₆ alkyl, C₁-C₆ alkoxy, halogen, hydroxy, amino, mono- or di-(C₁-C₆)alkylamino, nitro, halo(C₁-C₆)alkyl, carboxy, carboxamide, or —NH—C(O)—C₁-C₆ alkoxy; and R₁ R₂, and R₂₀ are as defined in Formula (I).

In another aspect, the invention provides a compound of Formula (VII), wherein R_{X} is H or CH₃; Y is hydrogen, halogen, C₁-C₁₀ alkyl, C₃-C₇ cycloalkyl(C₁-C₁₀)alkyl, or C₁-C₁₀ haloalkyl; V is —C₂-C₁₀ alkyl- optionally substituted with at least one group selected from C₁-C₆ alkyl, C₁-C₆ alkoxy, halogen, hydroxy, amino, mono- or di-(C₁-C₆)alkylamino, nitro, halo(C₁-C₆)alkyl, carboxy, carboxamide, or —NH—C(O)—C₁-C₆ alkoxy; or V is V₂-L-V₁; V₁ is —C₂-C₁₀ alkyl- optionally substituted with at least one group selected from C₁-C₆ alkyl, C₁-C₆ alkoxy, halogen, hydroxy, amino, mono- or di-(C₁-C₆)alkylamino, nitro, halo(C₁-C₆)alkyl, carboxy, carboxamide, or —NH—C(O)—C₁-C₆ alkoxy; L is —N(R_{N})C(O)—; V₂ is —C₂-C₁₀ alkyl- optionally substituted with at least one group selected from C₁-C₆ alkyl, C₁-C₆ alkoxy, halogen, hydroxy, amino, mono- or di-(C₁-C₆)alkylamino, nitro, halo(C₁-C₆)alkyl, carboxy, carboxamide, or —NH—C(O)—C₁-C₆ alkoxy; and R₁, R₂, and R₂₀ are as defined in Formula (I).

In another aspect, the invention provides a compound of Formula (VII), wherein R_{X} is H or CH₃; Y is hydrogen, halogen, C₁-C₁₀ alkyl, C₃-C₇ cycloalkyl(C₁-C₁₀)alkyl, or C₁-C₁₀ haloalkyl; V is —C₂-C₁₀ alkyl-; or V is V₂-L-V₁; V₁ is —C₁-C₁₀ alkyl-; L is —N(H)C(O)—; V₂ is —C₁-C₁₀ alkyl-; R₁ and R₂ are each —H; and R₂₀ is —H or —C(O)—C₁-C₆ alkoxy.

In another aspect, the invention provides a compound of Formula (VII) wherein the 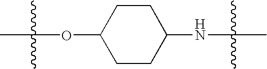
group is trans; R_{X} is H or CH₃; Y is C₁-C₁₀ alkyl, C₁-C₁₀ haloalkyl, or C₃-C₇ cycloalkyl(C₁-C₁₀)alkyl; R₁ and R₂ are independently H or C₁-C₆ alkyl; V is —C₂-C₁₀ alkyl- optionally substituted with 1 amino or —NH—C(O)—C₁-C₆ alkoxy group; or V is V₁—N(R_{N})C(O)—V₂; V₁ is —C₁-C₁₀ alkyl- optionally substituted with 1 amino or —NH—C(O)—C₁-C₆ alkoxy group; V₂ is —C₁-C₁₀ alkyl- optionally substituted with 1 amino or —NH—C(O)—C₁-C₆ alkoxy group; R_{N} is —H or C₁-C₆ alkyl; and R₂₀ is —H or —C(O)—C₁-C₆ alkoxy.

In another aspect, the invention provides a compound of Formula (VII) wherein the 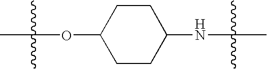
group is trans; R_{X} is H or CH₃; Y is methyl, ethyl, trifluoromethyl, or cyclopropylmethyl; R₁ and R₂ are H; V is —C₂-C₁₀ alkyl- optionally substituted with 1 amino or —NH—C(O)—C₁-C₆ alkoxy group; and R₂₀ is —H or —C(O)—C₁-C₆ alkoxy.

In another aspect, the invention provides a compound of Formula (VII) wherein the 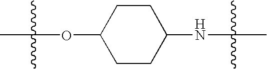
group is trans; R_{X} is H or CH₃; Y is methyl, ethyl, trifluoromethyl, or cyclopropylmethyl; R₁ and R₂ are H; V is V₁—N(R_{N})C(O)—V₂; V₁ is —C₁-C₁₀ alkyl- optionally substituted with 1 amino or —NH—C(O)—C₁-C₆ alkoxy group; V₂ is —C₁-C₁₀ alkyl- optionally substituted with 1 amino or —NH—C(O)—C₁-C₆alkoxy group; R_{N} is —H; and R₂₀ is —H or —C(O)—C₁-C₆ alkoxy.

In another aspect, the invention provides a method of treating inflammation comprising administering a therapeutically effective amount of a compound of Formula (VII), or a pharmaceutically acceptable salt thereof, to a patient in need of such treatment.

In another aspect, the invention provides a method of treating arthritis comprising administering a therapeutically effective amount of a compound of Formula (VII), or a pharmaceutically acceptable salt thereof, to a patient in need of such treatment.

In another aspect, the invention provides a method of treating angiogenesis comprising administering a therapeutically effective amount of a compound of Formula (VII), or a pharmaceutically acceptable salt thereof, to a patient in need of such treatment.

In another aspect, the invention provides a method of treating neurodegenerative disease comprising administering a therapeutically effective amount of a compound of Formula (VII), or a pharmaceutically acceptable salt thereof, to a patient in need of such treatment.

In another aspect, the invention provides a method of treating fungal infections comprising administering a therapeutically effective amount of a compound of Formula (VII), or a pharmaceutically acceptable salt thereof, to a patient in need of such treatment.

In another aspect, the invention provides a method of treating malaria comprising administering a therapeutically effective amount of a compound of Formula (VII), or a pharmaceutically acceptable salt thereof, to a patient in need of such treatment.

In another aspect, the invention provides a method of treating a disease or disorder related to cell proliferation comprising administering a therapeutically effective amount of a compound of Formula (VII), or a pharmaceutically acceptable salt thereof, to a patient in need of such treatment.

In another aspect, the invention provides a method of inhibiting cell proliferation comprising administering a therapeutically effective amount of a compound of Formula (VII), or a pharmaceutically acceptable salt thereof, to a patient in need of such treatment.

In another aspect, the invention provides a method of treating a disease or disorder related to Heat-shock protein 90 comprising administering a therapeutically effective amount of a compound of Formula (VII), or a pharmaceutically acceptable salt thereof, to a patient in need of such treatment.

In another aspect, the invention provides a method of inhibiting Heat-shock protein 90 comprising administering a therapeutically effective amount of a compound of Formula (VII), or a pharmaceutically acceptable salt thereof, to a patient in need of such treatment.

In another aspect, the invention provides a method of treating cancer comprising administering a therapeutically effective amount of a compound of Formula (VII), or a pharmaceutically acceptable salt thereof, to a patient in need of such treatment.

In another aspect, the invention provides a pharmaceutical composition comprising a therapeutically effective amount of a compound of Formula (VII), or a pharmaceutically acceptable salt thereof, and at least one pharmaceutically acceptable carrier, solvent, adjuvant or diluent.

In another aspect, the invention provides a method of treating Plasmodium falciparum comprising administering a therapeutically effective amount of a compound of Formula (VII) or a pharmaceutically acceptable salt thereof to a patient in need of such treatment.

In another aspect, the invention provides a compound of Formula (VIII) 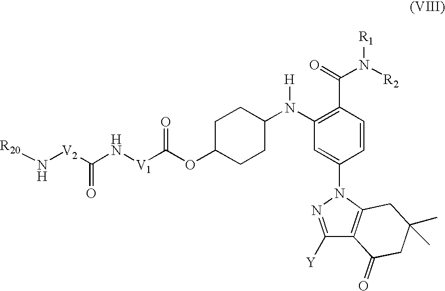
or a pharmaceutically acceptable salt thereof, wherein Y, V₁, V₂ R₁, R₂ and R₂₀ are as defined in Formula (I).

In another aspect, the invention provides a compound of Formula (VIII) wherein Y is hydrogen, halogen, C₁-C₁₀ alkyl, C₃-C₇ cycloalkyl(C₁-C₁₀)alkyl, or C₁-C₁₀ haloalkyl; V₁ is -C₁-C₁₀ alkyl- optionally substituted with 1 amino or —NH—C(O)—C₁-C₆ alkoxy group; V₂ is —C₁-C₁₀ alkyl optionally substituted with 1 amino or —NH—C(O)—C₁-C₆ alkoxy group; R₁ and R₂ are independently H or C₁-C₆ alkyl; and R₂₀ is —H or —C(O)—C₁-C₆ alkoxy.

In another aspect, the invention provides a compound of Formula (VIII) wherein the 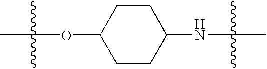
group is trans; Y is C₁-C₁₀ alkyl, C₁-C₁₀ haloalkyl, or C₃-C₇ cycloalkyl(C₁-C₁₀)alkyl; R₁ and R₂ are independently H or C₁-C₆ alkyl; V₁ is —C₁-C₁₀ alkyl- optionally substituted with 1 amino or —NH—C(O)—C₁-C₆ alkoxy group; V₂ is —C₁-C₁₀ alkyl- optionally substituted with 1 amino or —NH—C(O)—C₁-C₆ alkoxy group; and R₂₀ is —H or —C(O)—C₁-C₆ alkoxy.

In another aspect, the invention provides a compound of Formula (VIII) wherein the 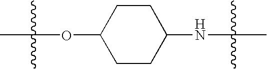
group is trans; Y is methyl, ethyl, trifluoromethyl, or cyclopropylmethyl; R₁ and R₂ are H; V₁ is —C₁-C₁₀ alkyl- optionally substituted with 1 amino or —NH—C(O)—C₁-C₆ alkoxy group; V₂ is —C₁-C₁₀ alkyl- optionally substituted with 1 amino or —NH—C(O)—C₁-C₆ alkoxy group; and R₂₀ is —H or —C(O)—C₁-C₆ alkoxy.

In another aspect, the invention provides a method of treating inflammation comprising administering a therapeutically effective amount of a compound of Formula (VIII), or a pharmaceutically acceptable salt thereof, to a patient in need of such treatment.

In another aspect, the invention provides a method of treating arthritis comprising administering a therapeutically effective amount of a compound of Formula (VIII), or a pharmaceutically acceptable salt thereof, to a patient in need of such treatment.

In another aspect, the invention provides a method of treating angiogenesis comprising administering a therapeutically effective amount of a compound of Formula (VIII), or a pharmaceutically acceptable salt thereof, to a patient in need of such treatment.

In another aspect, the invention provides a method of treating neurodegenerative disease comprising administering a therapeutically effective amount of a compound of Formula (VIII), or a pharmaceutically acceptable salt thereof, to a patient in need of such treatment.

In another aspect, the invention provides a method of treating fungal infections comprising administering a therapeutically effective amount of a compound of Formula (VIII), or a pharmaceutically acceptable salt thereof, to a patient in need of such treatment.

In another aspect, the invention provides a method of treating malaria comprising administering a therapeutically effective amount of a compound of Formula (VIII), or a pharmaceutically acceptable salt thereof, to a patient in need of such treatment.

In another aspect, the invention provides a method of treating a disease or disorder related to cell proliferation comprising administering a therapeutically effective amount of a compound of Formula (VIII), or a pharmaceutically acceptable salt thereof, to a patient in need of such treatment.

In another aspect, the invention provides a method of inhibiting cell proliferation comprising administering a therapeutically effective amount of a compound of Formula (VIII), or a pharmaceutically acceptable salt thereof, to a patient in need of such treatment.

In another aspect, the invention provides a method of treating a disease or disorder related to Heat-shock protein 90 comprising administering a therapeutically effective amount of a compound of Formula (VIII), or a pharmaceutically acceptable salt thereof, to a patient in need of such treatment.

In another aspect, the invention provides a method of inhibiting Heat-shock protein 90 comprising administering a therapeutically effective amount of a compound of Formula (VIII), or a pharmaceutically acceptable salt thereof, to a patient in need of such treatment.

In another aspect, the invention provides a method of treating cancer comprising administering a therapeutically effective amount of a compound of Formula (VIII), or a pharmaceutically acceptable salt thereof, to a patient in need of such treatment.

In another aspect, the invention provides a pharmaceutical composition comprising a therapeutically effective amount of a compound of Formula (VIII), or a pharmaceutically acceptable salt thereof, and at least one pharmaceutically acceptable carrier, solvent, adjuvant or diluent.

In another aspect, the invention provides a method of treating Plasmodium falciparum comprising administering a therapeutically effective amount of a compound of Formula (VIII) or a pharmaceutically acceptable salt thereof to a patient in need of such treatment.

In another aspect, the invention provides a compound of Formula (IX) 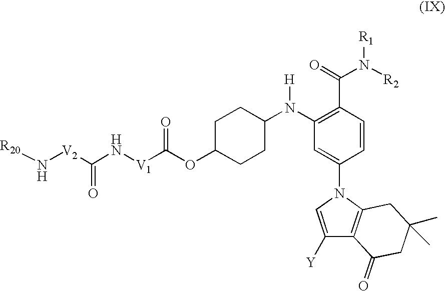
or a pharmaceutically acceptable salt thereof, wherein Y, V₁, V₂, R₁, R₂, and R₂₀ are as defined in Formula (I).

In another aspect, the invention provides a compound of Formula (IX) wherein Y is hydrogen, halogen, C₁-C₁₀ alkyl, C₃-C₇ cycloalkyl(C₁-C₁₀)alkyl, or C₁-C₁₀ haloalkyl; V₁ is —C₁-C₁₀ alkyl- optionally substituted with 1 amino or —NH—C(O)—C₁-C₆ alkoxy group; V₂ is —C₁-C₁₀ alkyl optionally substituted with 1 amino or —NH—C(O)—C₁-C₆ alkoxy group; R₁ and R₂ are independently H or C₁-C₆ alkyl;and R₂₀ is —H or —C(O)—C₁-C₆ alkoxy.

In another aspect, the invention provides a compound of Formula (IX) wherein the 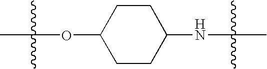
group is trans; Y is C₁-C₁₀ alkyl, C₁-C₁₀ haloalkyl, or C₃-C₇ cycloalkyl(C₁-C₁₀)alkyl; R₁ and R₂ are independently H or C₁-C₆ alkyl; V₁ is —C₁-C₁₀ alkyl- optionally substituted with 1 amino or —NH—C(O)—C₁-C₆ alkoxy group; V₂ is —C₁-C₁₀ alkyl- optionally substituted with 1 amino or —NH—C(O)—C₁-C₆ alkoxy group; and R₂₀ is —H or —C(O)—C₁-C₆ alkoxy.

In another aspect, the invention provides a compound of Formula (IX) wherein the 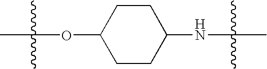
group is trans; Y is methyl, ethyl, trifluoromethyl, or cyclopropylmethyl; R₁ and R₂ are H; V₁ is —C₁-C₁₀ alkyl- optionally substituted with 1 amino or —NH—C(O)—C₁-C₆ alkoxy group; V₂ is —C₁-C₁₀ alkyl- optionally substituted with 1 amino or —NH—C(O)—C₁-C₆ alkoxy group; and R₂₀ is —H or —C(O)—C₁-C₆ alkoxy.

In another aspect, the invention provides a method of treating inflammation comprising administering a therapeutically effective amount of a compound of Formula (IX), or a pharmaceutically acceptable salt thereof, to a patient in need of such treatment.

In another aspect, the invention provides a method of treating arthritis comprising administering a therapeutically effective amount of a compound of Formula (IX), or a pharmaceutically acceptable salt thereof, to a patient in need of such treatment.

In another aspect, the invention provides a method of treating angiogenesis comprising administering a therapeutically effective amount of a compound of Formula (IX), or a pharmaceutically acceptable salt thereof, to a patient in need of such treatment.

In another aspect, the invention provides a method of treating neurodegenerative disease comprising administering a therapeutically effective amount of a compound of Formula (IX), or a pharmaceutically acceptable salt thereof, to a patient in need of such treatment.

In another aspect, the invention provides a method of treating fungal infections comprising administering a therapeutically effective amount of a compound of Formula (IX), or a pharmaceutically acceptable salt thereof, to a patient in need of such treatment.

In another aspect, the invention provides a method of treating malaria comprising administering a therapeutically effective amount of a compound of Formula (IX), or a pharmaceutically acceptable salt thereof, to a patient in need of such treatment.

In another aspect, the invention provides a method of treating a disease or disorder related to cell proliferation comprising administering a therapeutically effective amount of a compound of Formula (IX), or a pharmaceutically acceptable salt thereof, to a patient in need of such treatment.

In another aspect, the invention provides a method of inhibiting cell proliferation comprising administering a therapeutically effective amount of a compound of Formula (IX), or a pharmaceutically acceptable salt thereof, to a patient in need of such treatment.

In another aspect, the invention provides a method of treating a disease or disorder related to Heat-shock protein 90 comprising administering a therapeutically effective amount of a compound of Formula (IX), or a pharmaceutically acceptable salt thereof, to a patient in need of such treatment.

In another aspect, the invention provides a method of inhibiting Heat-shock protein 90 comprising administering a therapeutically effective amount of a compound of Formula (IX), or a pharmaceutically acceptable salt thereof, to a patient in need of such treatment.

In another aspect, the invention provides a method of treating cancer comprising administering a therapeutically effective amount of a compound of Formula (IX), or a pharmaceutically acceptable salt thereof, to a patient in need of such treatment.

In another aspect, the invention provides a pharmaceutical composition comprising a therapeutically effective amount of a compound of Formula (IX), or a pharmaceutically acceptable salt thereof, and at least one pharmaceutically acceptable carrier, solvent, adjuvant or diluent.

In another aspect, the invention provides a method of treating Plasmodium falciparum comprising administering a therapeutically effective amount of a compound of Formula (IX) or a pharmaceutically acceptable salt thereof to a patient in need of such treatment.

In another aspect, the invention provides a compound of Formula (X) 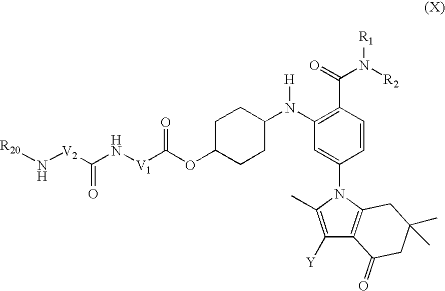
or a pharmaceutically acceptable salt thereof wherein Y, V₁, V₂, R₁, R₂ and R₂₀ are as defined in Formula (I).

In another aspect, the invention provides a compound of Formula (X) wherein Y is hydrogen, halogen, C₁-C₁₀ alkyl, C₃-C₇ cycloalkyl(C₁-C₁₀)alkyl, or C₁-C₁₀ haloalkyl; V₁ is —C₁-C₁₀ alkyl- optionally substituted with 1 amino or —NH—C(O)—C₁-C₆ alkoxy group; V₂ is —C₁-C₁₀ alkyl optionally substituted with 1 amino or —NH—C(O)—C₁-C₆ alkoxy group; R₁ and R₂ are independently H or C₁-C₆ alkyl;and R₂₀ is —H or —C(O)—C₁-C₆ alkoxy.

In another aspect, the invention provides a compound of Formula (X) wherein the 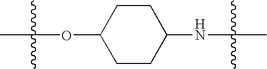
group is trans; Y is C₁-C₁₀ alkyl, C₁-C₁₀ haloalkyl, or C₃-C₇ cycloalkyl(C₁-C₁₀)alkyl; R₁ and R₂ are independently H or C₁-C₆ alkyl; V₁ is —C₁-C₁₀ alkyl- optionally substituted with 1 amino or —NH—C(O)—C₁-C₆ alkoxy group; V₂ is —C₁-C₁₀ alkyl- optionally substituted with 1 amino or —NH—C(O)—C₁-C₆ alkoxy group; and R₂₀ is —H or —C(O)—C₁-C₆ alkoxy.

In another aspect, the invention provides a compound of Formula (X) wherein the 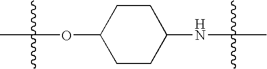
group is trans; Y is methyl, ethyl, trifluoromethyl, or cyclopropylmethyl; R₁ and R₂ are H; V₁ is —C₁-C₁₀ alkyl- optionally substituted with 1 amino or —NH—C(O)—C₁-C₆ alkoxy group; V₂ is —C₁-C₁₀ alkyl- optionally substituted with 1 amino or —NH—C(O)—C₁-C₆ alkoxy group; and R₂₀ is —H or —C(O)—C₁-C₆ alkoxy.

In another aspect, the invention provides a method of treating inflammation comprising administering a therapeutically effective amount of a compound of Formula (X), or a pharmaceutically acceptable salt thereof, to a patient in need of such treatment.

In another aspect, the invention provides a method of treating arthritis comprising administering a therapeutically effective amount of a compound of Formula (X), or a pharmaceutically acceptable salt thereof, to a patient in need of such treatment.

In another aspect, the invention provides a method of treating angiogenesis comprising administering a therapeutically effective amount of a compound of Formula (X), or a pharmaceutically acceptable salt thereof, to a patient in need of such treatment.

In another aspect, the invention provides a method of treating neurodegenerative disease comprising administering a therapeutically effective amount of a compound of Formula (X), or a pharmaceutically acceptable salt thereof, to a patient in need of such treatment.

In another aspect, the invention provides a method of treating fungal infections comprising administering a therapeutically effective amount of a compound of Formula (X), or a pharmaceutically acceptable salt thereof, to a patient in need of such treatment.

In another aspect, the invention provides a method of treating malaria comprising administering a therapeutically effective amount of a compound of Formula (X), or a pharmaceutically acceptable salt thereof, to a patient in need of such treatment.

In another aspect, the invention provides a method of treating a disease or disorder related to cell proliferation comprising administering a therapeutically effective amount of a compound of Formula (X), or a pharmaceutically acceptable salt thereof, to a patient in need of such treatment.

In another aspect, the invention provides a method of inhibiting cell proliferation comprising administering a therapeutically effective amount of a compound of Formula (X), or a pharmaceutically acceptable salt thereof, to a patient in need of such treatment.

In another aspect, the invention provides a method of treating a disease or disorder related to Heat-shock protein 90 comprising administering a therapeutically effective amount of a compound of Formula (X), or a pharmaceutically acceptable salt thereof, to a patient in need of such treatment.

In another aspect, the invention provides a method of inhibiting Heat-shock protein 90 comprising administering a therapeutically effective amount of a compound of Formula (X), or a pharmaceutically acceptable salt thereof, to a patient in need of such treatment.

In another aspect, the invention provides a method of treating cancer comprising administering a therapeutically effective amount of a compound of Formula (X), or a pharmaceutically acceptable salt thereof, to a patient in need of such treatment.

In another aspect, the invention provides a pharmaceutical composition comprising a therapeutically effective amount of a compound of Formula (X), or a pharmaceutically acceptable salt thereof, and at least one pharmaceutically acceptable carrier, solvent, adjuvant or diluent.

In another aspect, the invention provides a method of treating Plasmodium falciparum comprising administering a therapeutically effective amount of a compound of Formula (X) or a pharmaceutically acceptable salt thereof to a patient in need of such treatment.

In another aspect, the invention comprises a method of treating cancer comprising administering to a patient in need thereof a therapeutically effective amount of a compound of Formula (I) or a pharmaceutically acceptable salt thereof.

In another aspect, the invention provides a method of treating cancer comprising administering to a patient in need thereof a pharmaceutical composition comprising a therapeutically effective amount of a compound of Formula (I), or a pharmaceutically acceptable salt thereof.

In another aspect, the invention encompasses the use of a therapeutically effective amount of a compound or salt of Formula I for the preparation of a medicament for the treatment of cancer, inflammation, or arthritis in a patient in need of such treatment.

In another aspect, the invention encompasses a package comprising a compound or salt of Formula I in a container with instructions on how to use the compound.

In another aspect, the invention encompasses the use of a therapeutically effective amount of a compound or salt according of Formula I for the preparation of a medicament for the treatment of a disease or condition related to cell proliferation in a patient in need of such treatment.

In another aspect, the invention encompasses the use of a therapeutically effective amount of a compound or salt according of Formula I for the preparation of a medicament for the treatment of a disease or condition related to cell proliferation in a patient in need of such treatment, wherein the disease or condition is cancer, inflammation, or arthritis.

In another aspect, the invention encompasses the use of therapeutically effective amount of a compound or salt of Formula I for the preparation of a medicament for the treatment of a disease or disorder related to the activity of heat shock protein 90, in a subject in need of such.

In another aspect, the invention encompasses the use of therapeutically effective amount of a compound or salt of Formula I for the preparation of a medicament for the treatment of a disease or disorder related to the activity of heat shock protein 90, in a subject in need of such, wherein the HSP-90 mediated disorder is selected from the group of inflammatory diseases, infections, autoimmune disorders, stroke, ischemia, cardiac disorders, neurological disorders, fibrogenetic disorders, proliferative disorders, tumors, leukemias, neoplasms, cancers, carcinomas, metabolic diseases and malignant disease.

In another aspect, the invention encompasses the use of therapeutically effective amount of a compound or salt of Formula I for the preparation of a medicament for the treatment of a fibrogenetic disorder related to the activity of heat shock protein 90, in a subject in need of such, wherein the fibrogenetic disorder is selected from the group of scleroderma, polymyositis, systemic lupus, rheumatoid arthritis, liver cirrhosis, keloid formation, interstitial nephritis and pulmonary fibrosis.

In another aspect, the invention encompasses the use of a therapeutically effective amount of a compound or salt of Formula I for the preparation of a medicament for protecting a subject from infection caused by an organism selected from Plasmodium species.

In a preferred aspect, the invention encompasses the use of a therapeutically effective amount of a compound or salt of Formula I for the preparation of a medicament for protecting a subject from infection caused by Plasmodium falciparum.

In another aspect, the invention encompasses the use of a therapeutically effective amount of a compound or salt of Formula I for the preparation of a medicament for reducing the level of infection caused by an organism selected from Plasmodium species in a subject in need of such treatment.

In a preferred aspect, the invention encompasses the use of a therapeutically effective amount of a compound or salt of Formula I for the preparation of a medicament for reducing the level of infection caused by Plasmodium falciparum in a subject in need of such treatment

In another aspect, the invention encompasses the use of a therapeutically effective amount of a compound or salt of Formula I for the preparation of a medicament for treating a patient infected with a metazoan parasite.

In a preferred aspect, the invention encompasses the use of a therapeutically effective amount of a compound or salt of Formula I for the preparation of a medicament for treating a patient infected by a metazoan parasite which is Plasmodium falciparum.

The compounds of the invention have unique properties compared to the parent hydroxy compounds or their derivatives.

Definitions

The term “alkoxy” represents an alkyl group of indicated number of carbon atoms attached to the parent molecular moiety through an oxygen bridge. Examples of alkoxy groups include, for example, methoxy, ethoxy, propoxy and isopropoxy.

As used herein, the term “alkyl” includes those alkyl groups of a designed number of carbon atoms. Alkyl groups may be straight, or branched. Examples of “alkyl” include methyl, ethyl, propyl, isopropyl, butyl, iso-, sec- and tert-butyl, pentyl, hexyl, heptyl, 3-ethylbutyl, and the like.

The term “aryl” refers to an aromatic hydrocarbon ring system containing at least one aromatic ring. The aromatic ring may optionally be fused or otherwise attached to other aromatic hydrocarbon rings or non-aromatic hydrocarbon rings. Examples of aryl groups include, for example, phenyl, naphthyl, 1,2,3,4-tetrahydronaphthalene and biphenyl. Preferred examples of aryl groups include phenyl, naphthyl, and anthracenyl. More preferred aryl groups are phenyl and naphthyl. Most preferred is phenyl. The aryl groups of the invention may be substituted with various groups as provided herein. Thus, any carbon atom present within an aryl ring system and available for substitution may be further bonded to a variety of ring substituents, such as, for example, halogen, hydroxy, nitro, cyano, amino, C₁-C₈alkyl, C₁-C₈alkoxy, mono- and di(C₁-C₈alkyl)amino, C₃-C₁₀cycloalkyl, (C₃-C₁₀cycloalkyl)alkyl, (C₃-C₁₀cycloalkyl)alkoxy, C₂-C₉heterocycloalkyl, C₁-C₈alkenyl, C₁-C₈alkynyl, halo(C₁-C₈)alkyl, halo(C₁-C₈)alkoxy, oxo, amino(C₁-C₈)alkyl and mono- and di(C₁-C₈alkyl)amino(C₁-C₈)alkyl.

The term “cycloalkyl” refers to a C₃-C₈ cyclic hydrocarbon. Examples of cycloalkyl include cyclopropyl, cyclobutyl, cyclopentyl, cyclohexyl, cycloheptyl and cyclooctyl. More preferred are C₃-C₆ cycloalkyl groups. The cycloalkyl groups of the invention may be substituted with various groups as provided herein. Thus, any carbon atom present within a cycloalkyl ring system and available for substitution may be further bonded to a variety of ring substituents, such as, for example, halogen, hydroxy, nitro, cyano, amino, C₁-C₈alkyl, C₁-C₈alkoxy, mono- and di(C₁-C₈alkyl)amino, C₃-C₁₀cycloalkyl, (C₃-C₁₀cycloalkyl)alkyl, (C₃-C₁₀cycloalkyl)alkoxy, C₂-C₉heterocycloalkyl, C₁-C₈alkenyl, C₁-C₈alkynyl, halo(C₁-C₈)alkyl, halo(C₁-C₈)alkoxy, oxo, amino(C₁-C₈)alkyl and mono- and di(C₁-C₈alkyl)amino(C₁-C₈)alkyl.

The terms “halogen” or “halo” indicate fluorine, chlorine, bromine, and iodine.

The term “haloalkoxy” refers to an alkoxy group substituted with one or more halogen atoms, where each halogen is independently F, Cl, Br or I. Preferred halogens are F and Cl. Preferred haloalkoxy groups contain 1-6 carbons, more preferably 1-4 carbons, and still more preferably 1-2 carbons. “Haloalkoxy” includes perhaloalkoxy groups, such as OCF₃ or OCF₂CF₃. A preferred haloalkoxy group is trifluoromethoxy.

The term “haloalkyl” refers to an alkyl group substituted with one or more halogen atoms, where each halogen is independently F, Cl, Br or I. Preferred halogens are F and Cl. Preferred haloalkyl groups contain 1-6 carbons, more preferably 1-4 carbons, and still more preferably 1-2 carbons. “Haloalkyl” includes perhaloalkyl groups, such as CF₃ or CF₂CF₃. A preferred haloalkyl group is trifluoromethyl.

The term “heterocycloalkyl” refers to a ring or ring system containing at least one heteroatom selected from nitrogen, oxygen, and sulfur, wherein said heteroatom is in a non-aromatic ring. The heterocycloalkyl ring is optionally fused to or otherwise attached to other heterocycloalkyl rings and/or non-aromatic hydrocarbon rings and/or phenyl rings. Preferred heterocycloalkyl groups have from 3 to 7 members. More preferred heterocycloalkyl groups have 5 or 6 members. Examples of heterocycloalkyl groups include, for example, 1,2,3,4-tetrahydroisoquinolinyl, piperazinyl, morpholinyl, piperidinyl, tetrahydrofuranyl, pyrrolidinyl, pyridinonyl, and pyrazolidinyl. Preferred heterocycloalkyl groups include piperidinyl, piperazinyl, morpholinyl, pyrrolidinyl, pyridinonyl, dihydropyrrolidinyl, and pyrrolidinonyl. The heterocycloalkyl groups of the invention may be substituted with various groups as provided herein. Thus, any atom present within a heterocycloalkyl ring and available for substitution may be further bonded to a variety of ring substituents, such as, for example, halogen, hydroxy, nitro, cyano, amino, C₁-C₈alkyl, C₁-C₈alkoxy, mono- and di(C₁-C₈alkyl)amino, C₃-C₁₀cycloalkyl, (C₃-C₁₀cycloalkyl)alkyl, (C₃-C₁₀cycloalkyl)alkoxy, C₂-C₉heterocycloalkyl, C₁-C₈alkenyl, C₁-C₈alkynyl, halo(C₁-C₈)alkyl, halo(C₁-C₈)alkoxy, oxo, amino(C₁-C₈)alkyl and mono- and di(C₁-C₈alkyl)amino(C₁-C₈)alkyl.

The term “heteroaryl” refers to an aromatic ring system containing at least one heteroatom selected from nitrogen, oxygen, and sulfur. The heteroaryl ring may be fused or otherwise attached to one or more heteroaryl rings, aromatic or non-aromatic hydrocarbon rings or heterocycloalkyl rings. Examples of heteroaryl groups include, for example, pyridine, furan, thienyl, 5,6,7,8-tetrahydroisoquinoline and pyrimidines. The heteroaryl groups of the invention may be substituted with various groups as provided herein. Thus, any carbon atom present within an heteroaryl ring system and available for substitution may be further bonded to a variety of ring substituents, such as, for example, halogen, hydroxy, nitro, cyano, amino, C₁-C₈alkyl, C₁-C₈alkoxy, mono- and di(C₁-C₈alkyl)amino, C₃-C₁₀cycloalkyl, (C₃-C₁₀cycloalkyl)alkyl, (C₃-C₁₀cycloalkyl)alkoxy, C₂-C₉heterocycloalkyl, C₁-C₈alkenyl, C₁-C₈alkynyl, halo(C₁-C₈)alkyl, halo(C₁-C₈)alkoxy, oxo, amino(C₁-C₈)alkyl and mono- and di(C₁-C₈alkyl)amino(C₁-C₈)alkyl.

Preferred examples of heteroaryl groups include thienyl, benzothienyl, pyridyl, quinolyl, pyrazolyl, pyrimidyl, imidazolyl, benzimidazolyl, furanyl, benzofuranyl, dibenzofuranyl, thiazolyl, benzothiazolyl, isoxazolyl, oxadiazolyl, isothiazolyl, benzisothiazolyl, triazolyl, pyrrolyl, indolyl, pyrazolyl, and benzopyrazolyl.

The compounds of this invention may contain one or more asymmetric carbon atoms, so that the compounds can exist in different stereoisomeric forms. These compounds can be, for example, racemates, chiral non-racemic or diastereomers. In these situations, the single enantiomers, i.e., optically active forms, can be obtained by asymmetric synthesis or by resolution of the racemates. Resolution of the racemates can be accomplished, for example, by conventional methods such as crystallization in the presence of a resolving agent; chromatography, using, for example a chiral HPLC column; or derivatizing the racemic mixture with a resolving reagent to generate diastereomers, separating the diastereomers via chromatography, and removing the resolving agent to generate the original compound in enantiomerically enriched form. Any of the above procedures can be repeated to increase the enantiomeric purity of a compound.

When the compounds described herein contain olefinic double bonds or other centers of geometric asymmetry, and unless otherwise specified, it is intended that the compounds include the cis, trans, Z- and E-configurations. Likewise, all tautomeric forms are also intended to be included.

Pharmaceutical Compositions

The compounds of general Formula I may be administered orally, topically, parenterally, by inhalation or spray or rectally in dosage unit formulations containing conventional non-toxic pharmaceutically acceptable carriers, adjuvants and vehicles. The term parenteral as used herein includes percutaneous, subcutaneous, intravascular (e.g., intravenous), intramuscular, or intrathecal injection or infusion techniques and the like. In addition, there is provided a pharmaceutical formulation comprising a compound of general Formula I and a pharmaceutically acceptable carrier. One or more compounds of general Formula I may be present in association with one or more non-toxic pharmaceutically acceptable carriers and/or diluents and/or adjuvants, and if desired other active ingredients. The pharmaceutical compositions containing compounds of general Formula I may be in a form suitable for oral use, for example, as tablets, troches, lozenges, aqueous or oily suspensions, dispersible powders or granules, emulsion, hard or soft capsules, or syrups or elixirs.

Compositions intended for oral use may be prepared according to any method known in the art for the manufacture of pharmaceutical compositions and such compositions may contain one or more agents selected from the group consisting of sweetening agents, flavoring agents, coloring agents and preservative agents in order to provide pharmaceutically elegant and palatable preparations. Tablets contain the active ingredient in admixture with non-toxic pharmaceutically acceptable excipients that are suitable for the manufacture of tablets. These excipients may be for example, inert diluents, such as calcium carbonate, sodium carbonate, lactose, calcium phosphate or sodium phosphate; granulating and disintegrating agents, for example, corn starch, or alginic acid; binding agents, for example starch, gelatin or acacia, and lubricating agents, for example magnesium stearate, stearic acid or talc. The tablets may be uncoated or they may be coated by known techniques. In some cases such coatings may be prepared by known techniques to delay disintegration and absorption in the gastrointestinal tract and thereby provide a sustained action over a longer period. For example, a time delay material such as glyceryl monosterate or glyceryl distearate may be employed.

Formulations for oral use may also be presented as hard gelatin capsules, wherein the active ingredient is mixed with an inert solid diluent, for example, calcium carbonate, calcium phosphate or kaolin, or as soft gelatin capsules wherein the active ingredient is mixed with water or an oil medium, for example peanut oil, liquid paraffin or olive oil.

Formulations for oral use may also be presented as lozenges.

Aqueous suspensions contain the active materials in admixture with excipients suitable for the manufacture of aqueous suspensions. Such excipients are suspending agents, for example sodium carboxymethylcellulose, methylcellulose, hydropropyl-methylcellulose, sodium alginate, polyvinylpyrrolidone, gum tragacanth and gum acacia; dispersing or wetting agents may be a naturally-occurring phosphatide, for example, lecithin, or condensation products of an alkylene oxide with fatty acids, for example polyoxyethylene stearate, or condensation products of ethylene oxide with long chain aliphatic alcohols, for example heptadecaethyleneoxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyethylene sorbitan monooleate. The aqueous suspensions may also contain one or more preservatives, for example ethyl, or n-propyl p-hydroxybenzoate, one or more coloring agents, one or more flavoring agents, and one or more sweetening agents, such as sucrose or saccharin.

Oily suspensions may be formulated by suspending the active ingredients in a vegetable oil, for example arachis oil, olive oil, sesame oil or coconut oil, or in a mineral oil such as liquid paraffin. The oily suspensions may contain a thickening agent, for example beeswax, hard paraffin or cetyl alcohol. Sweetening agents and flavoring agents may be added to provide palatable oral preparations. These compositions may be preserved by the addition of an anti-oxidant such as ascorbic acid.

Dispersible powders and granules suitable for preparation of an aqueous suspension by the addition of water provide the active ingredient in admixture with a dispersing or wetting agent, suspending agent and one or more preservatives. Suitable dispersing or wetting agents or suspending agents are exemplified by those already mentioned above. Additional excipients, for example sweetening, flavoring and coloring agents, may also be present.

Pharmaceutical compositions of the invention may also be in the form of oil-in-water emulsions. The oily phase may be a vegetable oil or a mineral oil or mixtures of these. Suitable emulsifying agents may be naturally-occurring gums, for example gum acacia or gum tragacanth, naturally-occurring phosphatides, for example soy bean, lecithin, and esters or partial esters derived from fatty acids and hexitol, anhydrides, for example sorbitan monooleate, and condensation products of the said partial esters with ethylene oxide, for example polyoxyethylene sorbitan monooleate. The emulsions may also contain sweetening and flavoring agents.

Syrups and elixirs may be formulated with sweetening agents, for example glycerol, propylene glycol, sorbitol, glucose or sucrose. Such formulations may also contain a demulcent, a preservative and flavoring and coloring agents. The pharmaceutical compositions may be in the form of a sterile injectable aqueous or oleaginous suspension. This suspension may be formulated according to the known art using those suitable dispersing or wetting agents and suspending agents that have been mentioned above. The sterile injectable preparation may also be a sterile injectable solution or suspension in a non-toxic parentally acceptable diluent or solvent, for example as a solution in 1,3-butanediol. Among the acceptable vehicles and solvents that may be employed are water, Ringer's solution and isotonic sodium chloride solution. In addition, sterile, fixed oils are conventionally employed as a solvent or suspending medium. For this purpose any bland fixed oil may be employed including synthetic mono- or diglycerides. In addition, fatty acids such as oleic acid find use in the preparation of injectables.

The compounds of general Formula I may also be administered in the form of suppositories, e.g., for rectal administration of the drug. These compositions can be prepared by mixing the drug with a suitable non-irritating excipient that is solid at ordinary temperatures but liquid at the rectal temperature and will therefore melt in the rectum to release the drug. Such materials include cocoa butter and polyethylene glycols.

Compounds of general Formula I may be administered parenterally in a sterile medium. The drug, depending on the vehicle and concentration used, can either be suspended or dissolved in the vehicle. Advantageously, adjuvants such as local anesthetics, preservatives and buffering agents can be dissolved in the vehicle.

For disorders of the eye or other external tissues, e.g., mouth and skin, the formulations are preferably applied as a topical gel, spray, ointment or cream, or as a suppository, containing the active ingredients in a total amount of, for example, 0.075 to 30% w/w, preferably 0.2 to 20% w/w and most preferably 0.4 to 15% w/w. When formulated in an ointment, the active ingredients may be employed with either paraffinic or a water-miscible ointment base.

Alternatively, the active ingredients may be formulated in a cream with an oil-in-water cream base. If desired, the aqueous phase of the cream base may include, for example at least 30% w/w of a polyhydric alcohol such as propylene glycol, butane-1,3-diol, mannitol, sorbitol, glycerol, polyethylene glycol and mixtures thereof. The topical formulation may desirably include a compound which enhances absorption or penetration of the active ingredient through the skin or other affected areas. Examples of such dermal penetration enhancers include dimethylsulfoxide and related analogs. The compounds of this invention can also be administered by a transdermal device. Preferably topical administration will be accomplished using a patch either of the reservoir and porous membrane type or of a solid matrix variety. In either case, the active agent is delivered continuously from the reservoir or microcapsules through a membrane into the active agent permeable adhesive, which is in contact with the skin or mucosa of the recipient. If the active agent is absorbed through the skin, a controlled and predetermined flow of the active agent is administered to the recipient. In the case of microcapsules, the encapsulating agent may also function as the membrane. The transdermal patch may include the compound in a suitable solvent system with an adhesive system, such as an acrylic emulsion, and a polyester patch. The oily phase of the emulsions of this invention may be constituted from known ingredients in a known manner. While the phase may comprise merely an emulsifier, it may comprise a mixture of at least one emulsifier with a fat or an oil or with both a fat and an oil. Preferably, a hydrophilic emulsifier is included together with a lipophilic emulsifier which acts as a stabilizer. It is also preferred to include both an oil and a fat. Together, the emulsifier(s) with or without stabilizer(s) make-up the so-called emulsifying wax, and the wax together with the oil and fat make up the so-called emulsifying ointment base which forms the oily dispersed phase of the cream formulations. Emulsifiers and emulsion stabilizers suitable for use in the formulation of the invention include Tween 60, Span 80, cetostearyl alcohol, myristyl alcohol, glyceryl monostearate, and sodium lauryl sulfate, among others. The choice of suitable oils or fats for the formulation is based on achieving the desired cosmetic properties, since the solubility of the active compound in most oils likely to be used in pharmaceutical emulsion formulations is very low. Thus, the cream should preferably be a non-greasy, non-staining and washable product with suitable consistency to avoid leakage from tubes or other containers. Straight or branched chain, mono- or dibasic alkyl esters such as di-isoadipate, isocetyl stearate, propylene glycol diester of coconut fatty acids, isopropyl myristate, decyl oleate, isopropyl palmitate, butyl stearate, 2-ethylhexyl palmitate or a blend of branched chain esters may be used. These may be used alone or in combination depending on the properties required. Alternatively, high melting point lipids such as white soft paraffin and/or liquid paraffin or other mineral oils can be used.

Formulations suitable for topical administration to the eye also include eye drops wherein the active ingredients are dissolved or suspended in suitable carrier, especially an aqueous solvent for the active ingredients. The antiinflammatory active ingredients are preferably present in such formulations in a concentration of 0.5 to 20%, advantageously 0.5 to 10% and particularly about 1.5% w/w. For therapeutic purposes, the active compounds of this combination invention are ordinarily combined with one or more adjuvants appropriate to the indicated route of administration. If administered per os, the compounds may be admixed with lactose, sucrose, starch powder, cellulose esters of alkanoic acids, cellulose alkyl esters, talc, stearic acid, magnesium stearate, magnesium oxide, sodium and calcium salts of phosphoric and sulfuric acids, gelatin, acacia gum, sodium alginate, polyvinylpyrrolidone, and/or polyvinyl alcohol, and then tableted or encapsulated for convenient administration. Such capsules or tablets may contain a controlled-release formulation as may be provided in a dispersion of active compound in hydroxypropylmethyl cellulose. Formulations for parenteral administration may be in the form of aqueous or non-aqueous isotonic sterile injection solutions or suspensions. These solutions and suspensions may be prepared from sterile powders or granules having one or more of the carriers or diluents mentioned for use in the formulations for oral administration. The compounds may be dissolved in water, polyethylene glycol, propylene glycol, ethanol, corn oil, cottonseed oil, peanut oil, sesame oil, benzyl alcohol, sodium chloride, and/or various buffers. Other adjuvants and modes of administration are well and widely known in the pharmaceutical art.

Dosage levels of the order of from about 0.1 mg to about 140 mg per kilogram of body weight per day are useful in the treatment of the above-indicated conditions (about 0.5 mg to about 7 g per patient per day). The amount of active ingredient that may be combined with the carrier materials to produce a single dosage form will vary depending upon the host treated and the particular mode of administration. Dosage unit forms will generally contain between from about 1 mg to about 500 mg of an active ingredient. The daily dose can be administered in one to four doses per day. In the case of skin conditions, it may be preferable to apply a topical preparation of compounds of this invention to the affected area two to four times a day.

It will be understood, however, that the specific dose level for any particular patient will depend upon a variety of factors including the activity of the specific compound employed, the age, body weight, general health, sex, diet, time of administration, route of administration, and rate of excretion, drug combination and the severity of the particular disease undergoing therapy.

For administration to non-human animals, the composition may also be added to the animal feed or drinking water. It may be convenient to formulate the animal feed and drinking water compositions so that the animal takes in a therapeutically appropriate quantity of the composition along with its diet. It may also be convenient to present the composition as a premix for addition to the feed or drinking water. Preferred non-human animals include domesticated animals.

The compounds of the present invention may be prepared by use of known chemical reactions and procedures. Representative methods for synthesizing compounds of the invention are presented below. It is understood that the nature of the substituents required for the desired target compound often determines the preferred method of synthesis. All variable groups of these methods are as described in the generic description if they are not specifically defined below.

Methods of Preparation

General Procedure

Representative synthetic procedures for the preparation of compounds of the invention are outlined below in following schemes.

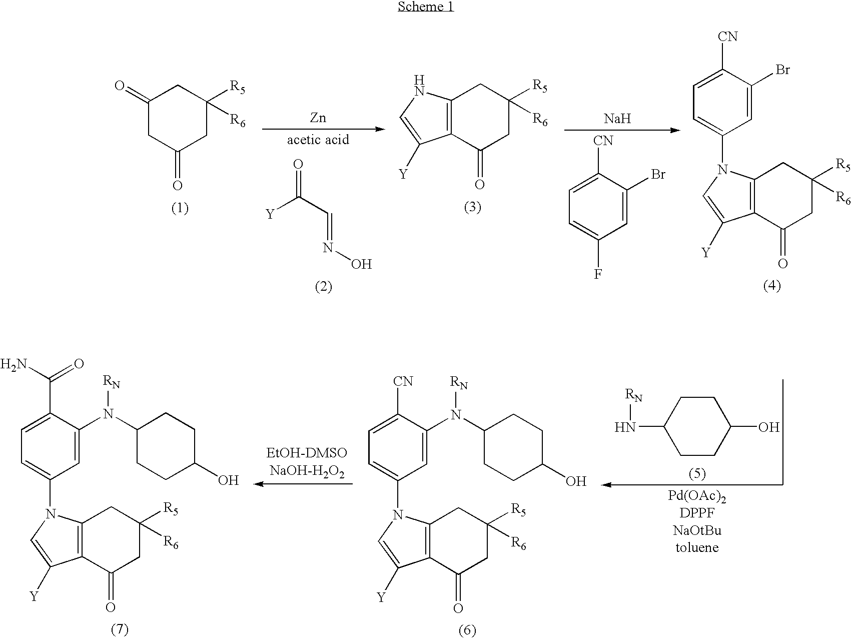

Compounds of formula (7), wherein Y is hydrogen, halogen, C₁-C₁₀ alkyl, C₁-C₁₀ haloalkyl, C₃-C₇ cycloalkyl, C₃-C₇ cycloalkyl(C₁-C₁₀)alkyl, C₁-C₆ acyl, aryl or heteroaryl and R_{N} is as defined in Formula (I), are prepared as described in Scheme 1. A dione of formula (1), where R₅ and R₆ are as defined in Formula (I), is treated with Zinc, an aqueous acid, and a compound of formula (2) to provide a compound of formula (3). A compound of formula (3) is treated with a base and 2-bromo-4-fluorobenzonitrile in an appropriate solvent to provide a compound of formula (4). A compound of formula (4) is treated with an amino cyclohexanol of formula (5), a metal, optionally a ligand, and a base, in an appropriate solvent, with heating or microwaving to provide a compound of formula (6). A compounds of formula (6) is treated with hydrogen peroxide and aqueaous sodium hydroxide to provide a compound of formula (7).

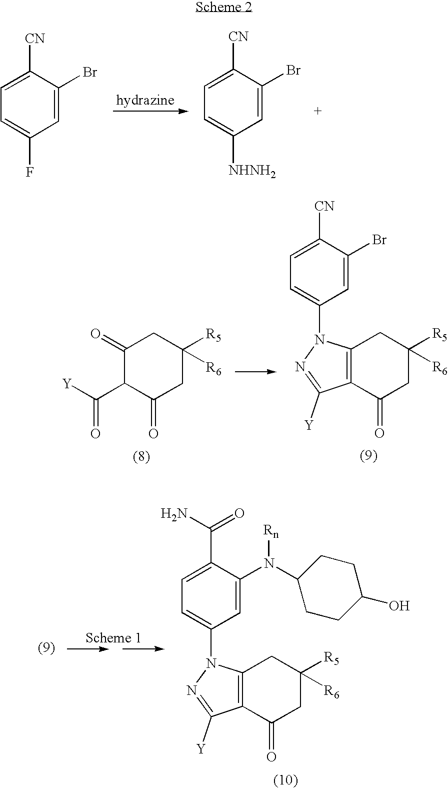

Compounds of formula (10), wherein Y is hydrogen, halogen, C₁-C₁₀ alkyl, C₁-C₁₀ haloalkyl, C₃-C₇ cycloalkyl, C₃-C₇ cycloalkyl(C₁-C₁₀)alkyl, C₁-C₆ acyl, aryl or heteroaryl and R_{N} is as defined in Formula (I), are prepared as described in Scheme 2. 2-Bromo-4-fluorobenzonitrile, in an appropriate solvent, is treated with hydrazine to provide 2-bromo-4-hydrazinylbenzonitrile. 2-Bromo-4-hydrazinylbenzonitrile is treated with a compound of formula (8), in an appropriate solvent, and heated or microwaved to provide a compound of formula (9). A compound of formula (9) is treated as described in Scheme 1 to provide a compound of formula (10).

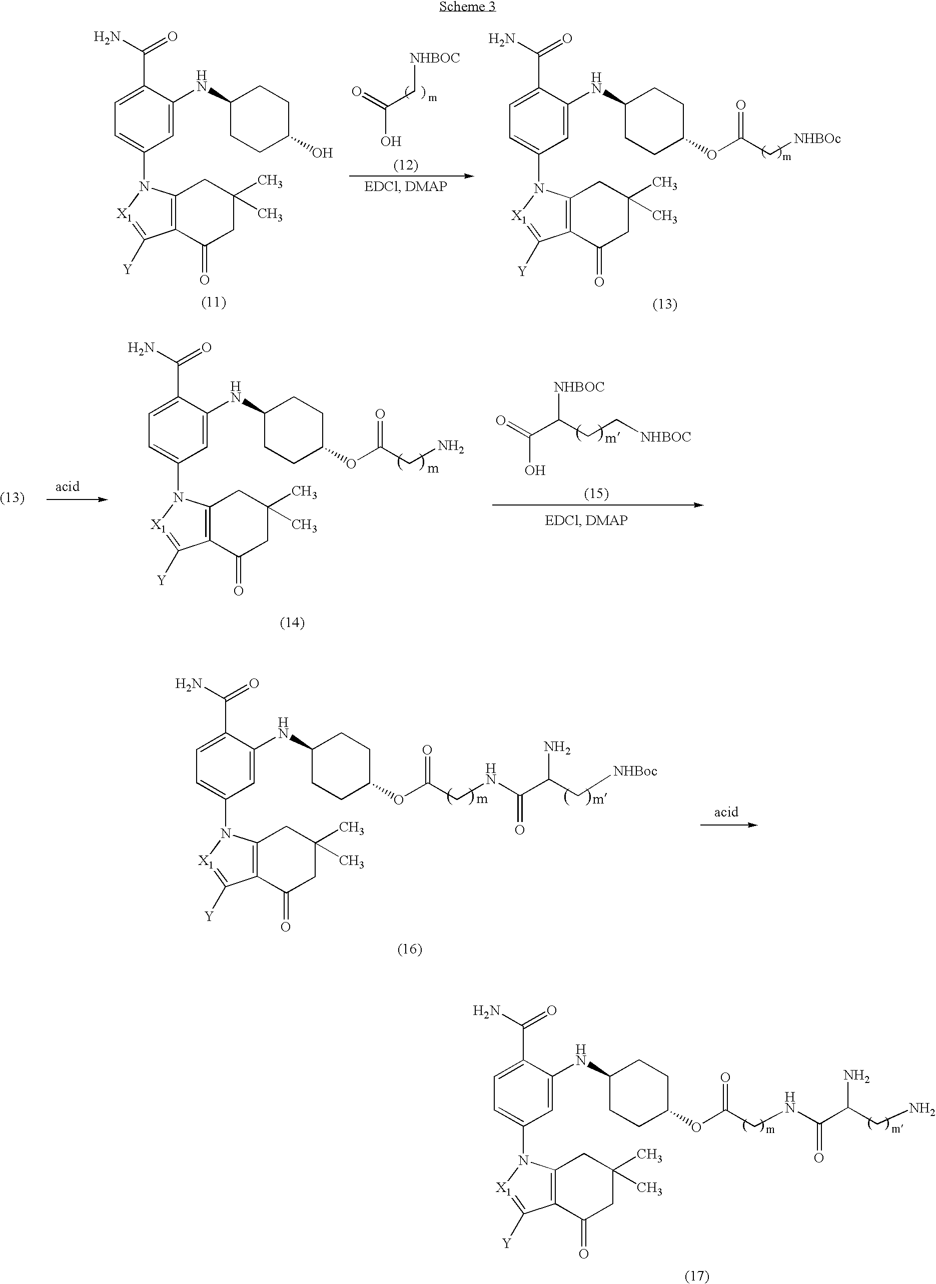

Compounds of formula (16), wherein X₁, Y, m, and m′ are as defined if Formula (I), are prepared as described in Scheme 3. A compound of formula (11), essentially prepared as described in the Examples/Schemes contained herein, is treated with an acid of formula (12), DMAP, and carbodiimide in an appropriate solvent to provide a compound of formula (13). Suitable carbodiimides include, but are not limited to, 1,3-dicyclohexylcarbodiimide (DCC), 1-cyclohexyl-3-(2-morpholinoethyl)carbodiimide metho-p-toluenesulfonate, and 1-(3-dimethylaminopropyl)-3-ethylcarbodiimied hydrochloride (EDCI). A compounds of formula (13) is treated with an acid in an appropriate solvent to provided a compound of formula (14). A compound of formula (14) is treated with an acid of formula (15), DMAP, and a carbodiimide to provide a compound of formula (16). A compound of formula (16) is treated with an acid in an appropriate solvent to provide a compound of formula (17).

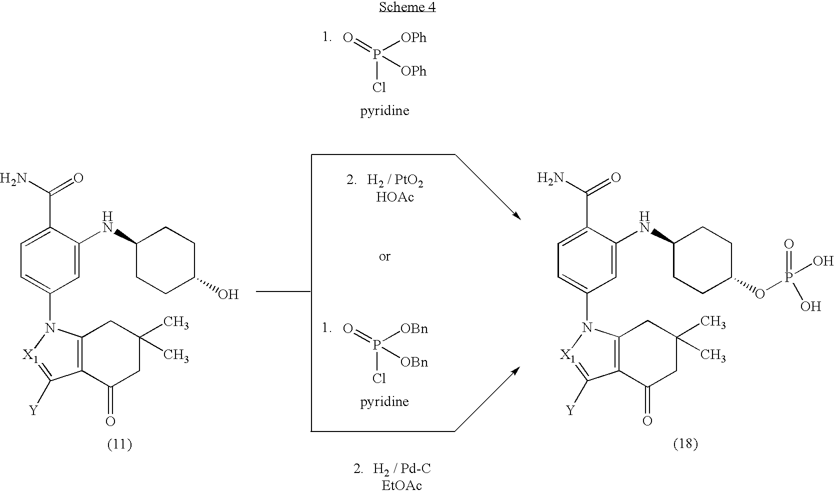

Compounds of formula (18), wherein X₁ and Y are as defined in Formula (I), are prepared as described in Scheme 4. A compound of formula (11), prepared as described in the Examples/Schemes contained herein, is treated with an O-protected phosphoryl chloride in an appropriate solvent to provide the phosphonate which is deprotected under the appropriate conditions to provide the acid. It is to be understood that the mono or di-salt is prepared using conditions well known to those of skill in the art. For example, acids of formula (12) can be treated with 1 or 2 equivalents of sodium hydride to provide the corresponding mono-sodium or di-sodium salt. Potassium hydride provides the potassium salt.

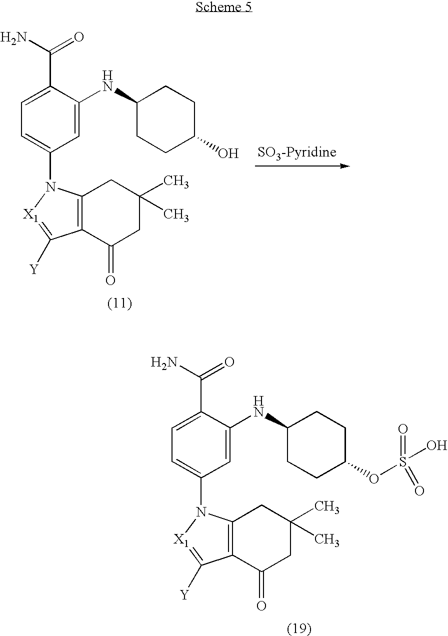

Compounds of formula (19), wherein X₁ and Y are as defined Formula (I), are prepared as described in Scheme 5. A compound of formula (11), prepared as described in the Examples/Schemes contained herein, is treated with a sulfur trioxide complex to provide a sulfonate of formula (19). It is to be understood that the salt is prepared using conditions well known to those of skill in the art. For example, acids of formula (19) can be treated with sodium hydride or sodium hydroxide to provide the corresponding sodium salt. Potassium hydride/hydroxide provides the potassium salt.

Those having skill in the art will recognize that the starting materials and reaction conditions may be varied, the sequence of the reactions altered, and additional steps employed to produce compounds encompassed by the present invention, as demonstrated by the following examples. In some cases, protection of certain reactive functionalities may be necessary to achieve some of the above transformations. In general, the need for such protecting groups as well as the conditions necessary to attach and remove such groups will be apparent to those skilled in the art of organic synthesis.

The disclosures of all articles and references mentioned in this application, including patents, are incorporated herein by reference in their entirety.

Structures were named using ChemDraw version 10.0, which is available from Cambridgesoft.com in Cambridge, Mass.

### EXAMPLES

The preparation of intermediates and compounds of the invention is illustrated further by the following examples, which are not to be construed as limiting the invention in scope or spirit to the specific procedures and compounds described in them. In all cases, unless otherwise specified, the column chromatography is performed using a silica gel solid phase.

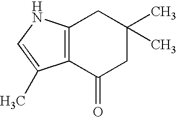

### Example 1

### 3,6,6-Trimethyl-1,5,6,7-tetrahydro-indol-4-one

To a solution of anti-pyruvic aldehyde-1-oxime (10 g, 1 eq) and 5,5-dimethyl-1,3-cyclohexanedione (16.1 g, 1 eq) in HOAc-H₂O (7:3, 200 mL), was added zinc powder (14.95 g, 2 eq) slowly cooling with a water bath at room temperature. The mixture was refluxed overnight, concentrated to dryness, partitioned between brine (300 mL) and dichloromethane (300 mL). The pH was adjusted to ca. 6 with saturated aqueous NaHCO₃, then extracted with dichloromethane (3×200 mL). The organic layers were combined, dried over Na₂SO₄, filtered, concentrated to give crude product. This was purified by flash chromatography, eluting with 5% ethyl acetate in dichloromethane to give expected product, which was triturated in ether-hexane (2:1) for 1 hour, then filtered, washed with hexane to give pure title compound (9 g, 45% yield) as a solid.

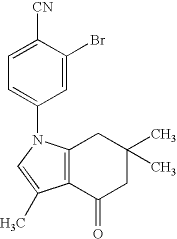

### Example 2

### 2-Bromo-4-(3,6,6-trimethyl-4-oxo-4,5,6,7-tetrahydro-indol-1-yl)-benzonitrile

The title compound of Example 1 (9.8 g, 55.3 mmol) and 2-bromo-4-fluorobenzonitrile (13.27 g, 66.4 mmol) were dissolved in anhydrous dimethylformamide (DMF, 300 mL). To this was added sodium hydride (NaH, 95%, 2.79 g, 111 mmol) and the reaction was stirred at 55° C. for 1 hour. The reaction mixture was cooled to room temperature and water was added. A tan solid precipitated which was filtered, washed with water and ether and then dried in vacuo (16.5 g, 84%). MS m/z: (M+H)=358.1.

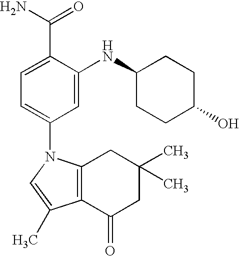

### Example 3

### 2-(trans-4-hydroxycyclohexylamino)-4-(3,6,6-trimethyl-4-oxo-4,5,6,7-tetrahydro-1H-indol-1-yl)benzamide

A “Personal Chemistry” microwave vial was charged with the title compound of Example 2 [2-Bromo-4-(3,6,6-trimethyl-4-oxo-4,5,6,7-tetrahydro-indol-1-yl)-benzonitrile (1.072 g, 3.0 mmol)], trans-4-aminocyclohexanol (1.382 g, 12.0 mmol), palladium (II) acetate (33.7 mg, 5 mol %), 1,1′-bis(diphenylphosphino)ferrocene (166.3 mg, 10 mol %), and sodium tert-butoxide (576.7 mg, 6.0 mmol). To this was added toluene (20 mL) and the reaction was heated with microwave irradiation to a temperature of 115° C. for fifteen minutes. After allowing the reaction vessel to cool, a suspension formed and was filtered and the filtrate evaporated. The residue was purified by flash chromatography. The intermediate product was hydrolyzed by dissolution in 25% dimethylsulfoxide/ethanol, adding 0.5 mL of 1N sodium hydroxide and 0.5 mL of 30% aqueous hydrogen peroxide, followed by stirring at room temperature for 4 hours. After judging the reaction to be complete by TLC, the DMSO/ethanol mixture was diluted with water and extracted with ethyl acetate (3×). The combined organics were washed with brine (2×), dried over Na₂SO₄, and evaporated. The compound was purified by column chromatography eluting with EtOAc-MeOH to yield 575 mg (47% yield) of the title compound as a white powder. MS m/z: (M+H)=410.3

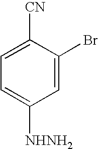

### Example 4

### 3-bromo-4-cyanophenylhydrazine

In a clean, dry 250-mL round-bottom flask, 2-bromo-4-fluorobenzonitrile (25.34 g) was dissolved in tetrahydrofuran (50 mL) under N₂. To this was slowly added anhydrous hydrazine (50 mL). The solution color changed from yellow to red-orange. The reaction was allowed to stir at room temperature for 16 hours. A yellow-white crystalline solid precipitated from the solution. The mixture was then diluted with THF (50 mL) to dissolve the solids. The organic layer was then washed with saturated sodium bicarbonate solution until the pH of the organic layer was approximately 8.5. The organic layer was isolated and the solvent was removed under reduced pressure to give a white solid. This was place in a fritted glass funnel and washed with 1.5 L of water, followed by of diethyl ether (ca. 200 mL). The ether wash was then combined with the white solid and dried under reduced pressure. The title compound was isolated as a fluffy, white or off-white solid (23.43 g, 87.20% yield). MS m/z: calculated=212.05; m/z observed=252.98 (M+41).

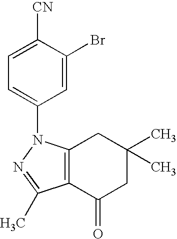

### Example 5

### 2-Bromo-4-(3,6,6-trimethyl-4-oxo-4,5,6,7-tetrahydro-indazol-1-yl)-benzonitrile

In a clean, dry 20 mL microwave reaction vial, the title compound of Example 4 (2.49 g) was combined with 2-acetyl-5,5-dimethyl-1,3-cyclohexanedione (2.14 g). The contents of the vial were dissolved in ethanol-acetic acid (12 mL, 3:1). The vial was sealed and agitated on a vortex. The vial was then placed in the microwave reactor and heated to 150° C. for 15 minutes (900 s), fixed-hold-time on, absorbance high. The reaction was stirred 15 s prior to irradiation. The vial was then cooled by forced air and then placed in the refrigerator for 1 hour. The cooled solution was then diluted with of water (8 mL) and poured onto a fritted glass funnel. The orange solid was washed with of H₂O (100 mL) followed by of ethanol (25 mL). The solid was then dried under reduced pressure. The title compound was obtained as of a light-orange crystalline solid (3.7463 g, 88.85% yield). MS m/z=358.1.

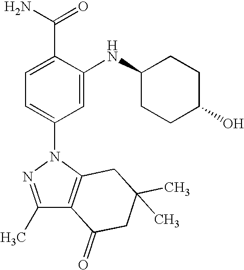

### Example 6

### 2-(trans-4-hydroxycyclohexylamino)-4-(3,6,6-trimethyl-4-oxo-4,5,6,7-tetrahydro-1H-indazol-1-yl)benzamide

1-(2-Bromo-4-cyanophen-4-yl)-3,6,6-trimethyltetrahydroindazal-4-one (2.0 g, 5.6 mmol), Pd(OAc)₂ (64 mg, 5 mol %), DPPF (312 mg, 10 mol %) and NaO^{t}Bu (1.08 g, 11.2 mmol) were added to a 20 mL microwave vial. Toluene (15 mL) and trans-4-aminocyclohexanol (1.29 g, 2 mol eq) were added and the vial was evacuated and back-filled with N₂. The reaction mixture was heated at 130° C. for 20 minutes (microwave). After cooling the reaction mixture was filtered and the solids washed with EtOAc. The product acetate ester was purified using a Biotage SP1 Flash chromatography system (Biotage Si 12+M™, TLC Method, eluting with hexanes and ethyl acetate). Acetate product was recovered as an off-white solid (1.3 g, 54%); LC/MS (m/z): M+H=435.3.

The preceding product (1.3 g, 3.0 mmol) was dissolved in ethanol (6 mL) and DMSO (3 mL) to which NaOH (5 N, 1.2 mL, 2 mol eq) and H₂O₂ (large excess, 30% solution in H₂O) were added. The reaction mixture was stirred at room temperature for 1 hour. The reaction mixture was washed with H₂O and extracted with EtOAc. The product was purified using a Biotage SP1 Flash chromatography system (Biotage Si 12+M™, TLC Method, eluting with CH₂Cl₂ and CMA 80). The title compound was obtained as an off-white solid (1.1 g, 84%); LC/MS (m/z): M+H=411.2.

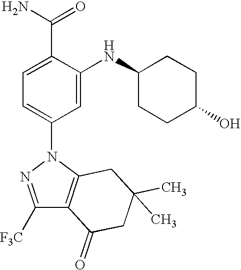

### Example 7

### 4-(6,6-dimethyl-4-oxo-3-(trifluoromethyl)-4,5,6,7-tetrahydro-1H-indazol-1-yl)-2-((1r,4r)-4-hydroxycyclohexylamino)benzamide

2-Bromo-4-(6,6-dimethyl-4-oxo-3-trifluoromethyl-4,5,6,7-tetrahydro-indazol-1-yl)-benzonitrile (60 mg, 0.15 mmol), Pd(OAc)₂ (1.7 mg, 5 mol %), DPPF (8.5 mg, 10 mol %) and NaO^{t}Bu (29.4 mg, 0.3 mmol) were added to a microwave vial. Toluene (0.5 mL) and trans-4-aminocyclohexanol (34 mg, 2 mol eq) were added and the vial was evacuated and back-filled with N₂. The reaction mixture was heated at 130° C. for 20 minutes (microwave). The reaction mixture was filtered and the solids washed with CH₂Cl₂. The product was purified using a Biotage SP1 Flash chromatography system (Biotage Si 12+M™, TLC Method, eluting with CH₂Cl₂ and CMA 80). The product pyrazole (10 mg, 2.2×10⁻⁵ mol) was dissolved in ethanol (0.8 mL) and DMSO (0.2 mL) to which NaOH (5 N, 9 μL, 2 mol eq) and H₂O₂ (excess, 30% solution in H₂O) was added. The reaction mixture was stirred at room temperature for 1 hour. The reaction mixture was washed with H₂O and extracted with EtOAc. The product was purified using a Biotage SP1 Flash chromatography system (Biotage Si 12+M™, TLC Method, eluting with CH₂Cl₂ and CMA 80). The title compound was obtained as an off-white solid (10mg, 96% yield), LC/MS (m/z): M+H=465.2.

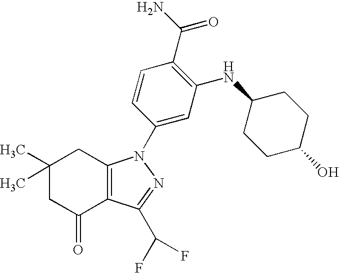

### Example 8

### 4-(3-(difluoromethyl)-6,6-dimethyl-4-oxo-4,5,6,7-tetrahydro-1H-indazol-1-yl)-2-(trans-4-hydroxycyclohexylamino)benzamide

2-Bromo-4-(3-difluoromethyl-6,6-dimethyl-4-oxo-4,5,6,7-tetrahydro-indazol-1-yl)-benzonitrile (394.2 mg, 1.0 mmol), trans-4-aminocyclohexanol (288.0 mg mg, 2.5 mmol, 2.5 eq.), palladium (II) acetate (11.2 mg, 5 mol %), 1,1′-bis(diphenylphosphino)ferrocene (55.4 mg, 10 mol %), and sodium tert-butoxide (192.2 mg, 2.0 mmol, 2.0 eq.) were suspended in 4 mL of toluene. The reaction mixture was microwaved at 120° C. for 20 minutes. After cooling the reaction mixture, the solvent was removed in vacuo, and the residue diluted with water. The aqueous suspension was extracted with EtOAc (×3) and the combined organics washed with brine, dried over Na₂SO₄, and concentrated under reduced pressure. The intermediate nitrile and the desired amide (along with their O-acetyl adducts) were collected and combined after purifying by column chromatography (CH₂Cl₂/CMA 80), and were dissolved in 20 mL of 4:1 EtOH-DMSO. 1.2 mL of 1 N NaOH and 0.5 mL of 30% H₂O₂ were added, and the reaction mixture stirred at room temperature for 2 hours. The solution was diluted with water, extracted into EtOAc (×3), and the combined organics washed with brine (×2), dried over Na₂SO₄, and concentrated under reduced pressure. The residue was purified by column chromatography (EtOAc/MeOH), yielding 165.2 mg (37.0% yield) of the title compound as a pale yellow foam.

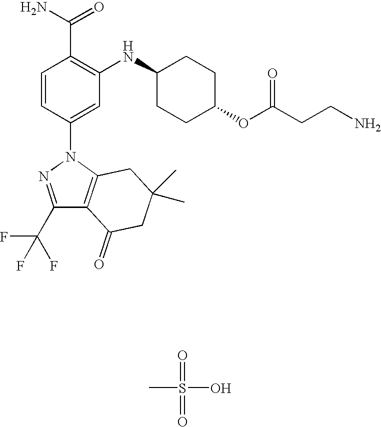

### Example 9

### trans-4-(2-carbamoyl-5-(6,6-dimethyl-4-oxo-3-(trifluoromethyl)-4,5,6,7-tetrahydro-1H-indazol-1-yl)phenylamino)cyclohexyl 3-aminopropanoate methanesulfonate

4-(6,6-Dimethyl-4-oxo-3-trifluoromethyl-4,5,6,7-tetrahydro-indazol-1-yl)-2-(trans-4-hydroxy-cyclohexylamino)-benzamide (6.9 g, 14.862 mmol), Boc-β-Ala-OH (5.624 g, 29.724 mmol, 2.0 equiv), N-(3-Dimethylaminopropyl)-N-ethylcarbodiimide hydrochloride (5.698 g, 29.724 mmol, 2.0 equiv), and 4-Dimethylaminopyridine (catalytic amount) were dissolved in 300 mL of CH₂Cl₂ and stirred at room temperature for sixteen hours. The solvent was removed in vacuo and the resultant residue purified by column chromatography (EtOAc/hexanes) to yield 3-tert-Butoxycarbonylamino-propionic acid 4-[2-carbamoyl-5-(6,6-dimethyl-4-oxo-3-trifluoromethyl-4,5,6,7-tetrahydro-indazol-1-yl)-phenylamino]-cyclohexyl ester as a white foam (7.486 grams, 94.1% yield). This material was then dissolved in 50 mL of CH₂Cl₂ and cooled to 0° C. in an ice bath. Fifty mL of trifluoroacetic acid was then added and the reaction stirred at 0° C. for 10 minutes and at room temperature for one hour. The solvent was removed in vacuo and the residue diluted with 250 mL of H₂O and neutralized by addition of NaHCO₃. The aqueous mixture was extracted with EtOAc ×3 and the combined organic extracts washed with brine ×2, dried over Na₂SO₄, and concentrated in vacuo to yield 3-Amino-propionic acid 4-[2-carbamoyl-5-(6,6-dimethyl-4-oxo-3-trifluoromethyl-4,5,6,7-tetrahydro-indazol-1-yl)-phenylamino]-cyclohexyl ester as an off-white solid (7.09 g, 89.1% yield). The methane sulfonate was formed by dissolving in 1:1 CH₂Cl₂:MeOH, adding one equivalent of CH₃SO₃H, stirring for one hour, and concentrating in vacuo.

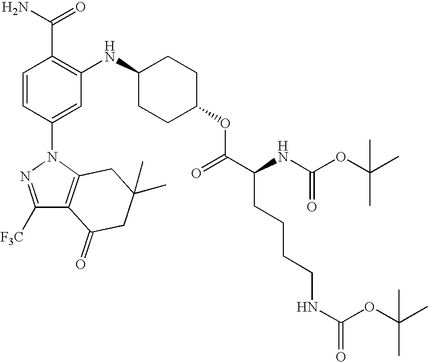

### Example 10

### (S)-(trans-4-(2-carbamoyl-5-(6,6-dimethyl-4-oxo-3-(trifluoromethyl)-4,5,6,7-tetrahydro-1H-indazol-1-yl)phenylamino)cyclohexyl) 2,6-bis(tert-butoxycarbonylamino)hexanoate

trans-4-(6,6-Dimethyl-4-oxo-3-trifluoromethyl-4,5,6,7-tetrahydro-indazol-1-yl)-2-(4-hydroxy-cyclohexylamino)-benzamide (0.57 mmol, 266 mg), N^{60, 68} -bis-BOC-L-lysine (0.87, 300 mg), 4-dimethylaminopyridine (17 mg), and EDC (1.14 mmol, 219 mg) were combined and diluted with methylene chloride (15 mL). After 18 hours of stirring, the mixture was concentrated and subjected to silica gel chromatography, affording the desired ester as a white foam (421 mg, 93%).

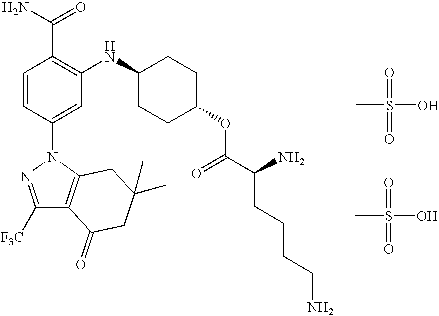

### Example 11

### (S)-(trans-4-(2-carbamoyl-5-(6,6-dimethyl-4-oxo-3-(trifluoromethyl)-4,5,6,7-tetrahydro-1H-indazol-1-yl)phenylamino)cyclohexyl)2,6-diaminohexanoate dimethanesulfonate

The product of Example 10 (0.48 mmol, 385 mg) was dissolved in methylene chloride (10 mL), cooled to 0 degrees Celsius, and treated with trifluoroacetic acid (10 mL). After 30 minutes, the reaction was complete. After concentration in vacuo, the residue was diluted with a mixture of methanol (6 mL) and methylene chloride (2 mL). Methanesulphonic acid (1 mmol, 0.065 mL) was added. After 5 minutes, the reaction was concentrated, azeotroped with dry ethanol, and triturated once with dry diethyl ether, affording the desired lysine ester as a hygroscopic white solid (500 mg, ca. quant.)

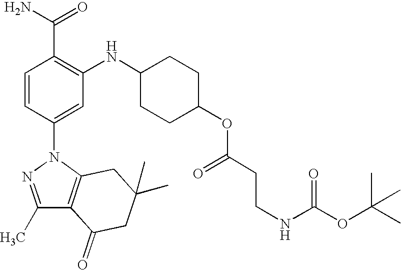

### Example 12

### 4-(2-carbamoyl-5-(3,6,6-trimethyl-4-oxo-4,5,6,7-tetrahydro-1H-indazol-1-yl)phenylamino)cyclohexyl 3-(tert-butoxycarbonylamino)propanoate

2-(4-Hydroxy-cyclohexylamino)-4-(3,6,6-trimethyl-4-oxo-4,5,6,7-tetrahydro-indazol-1-yl)-benzamide (250 mg, 0.609 mmol), Boc-β-Ala-OH (230.4 mg, 1.218 mmol, 2.0 equiv), N-(3-Dimethylaminopropyl)-N-ethylcarbodiimide hydrochloride (233.5 mg g, 1.218 mmol, 2.0 equiv), and 4-Dimethylaminopyridine (catalytic amount) were dissolved in 20 mL of CH₂Cl₂ and stirred at room temperature for sixteen hours. The solvent was removed in vacuo and the resultant residue purified by column chromatography (EtOAc/hexanes) to yield 3-tert-Butoxycarbonylamino-propionic acid 4-[2-carbamoyl-5-(3,6,6-trimethyl-4-oxo-4,5,6,7-tetrahydro-indazol-1-yl)-phenylamino]-cyclohexyl ester as a white foam (346.0 mg, 97.8% yield).

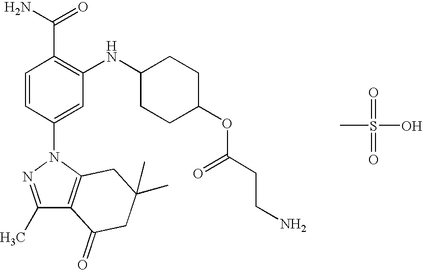

### Example 13

### 4-(2-carbamoyl-5-(3,6,6-trimethyl-4-oxo-4,5,6,7-tetrahydro-1H-indazol-1-yl)phenylamino)cyclohexyl 3-aminopropanoate methanesulfonate

4-(2-carbamoyl-5-(3,6,6-trimethyl-4-oxo-4,5,6,7-tetrahydro-1H-indazol-1-yl)phenylamino)cyclohexyl 3-(tert-butoxycarbonylamino)propanoate was dissolved in 4 mL of CH₂Cl₂ and cooled to 0° C. in an ice bath. Four mL of trifluoroacetic acid was then added and the reaction stirred at 0° C. for 10 minutes and at room temperature for one hour. The solvent was removed in vacuo and the residue diluted with H₂O and neutralized by addition of saturated aqueous sodium bicarbonate. The aqueous mixture was extracted with EtOAc ×3 and the combined organic extracts washed with brine ×2, dried over Na₂SO₄, and concentrated in vacuo to yield 3-Amino-propionic acid 4-[2-carbamoyl-5-(3,6,6-trimethyl-4-oxo-4,5,6,7-tetrahydro-indazol-1-yl)-phenylamino]-cyclohexyl ester as an off-white solid (277.2 mg, 94.5% yield). The methane sulfonate was formed by dissolving in 1:1 CH₂Cl₂:MeOH, adding one equivalent of CH₃SO₃H, stirring for one hour, and concentrating in vacuo. LCMS M+1=482.5

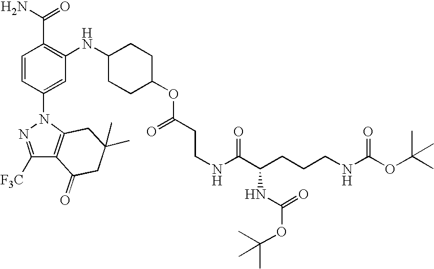

### Example 14

### (S)-4-(2-carbamoyl-5-(6,6-dimethyl-4-oxo-3-(trifluoromethyl)-4,5,6,7-tetrahydro-1H-indazol-1-yl)phenylamino)cyclohexyl 3-(2,5-bis(tert-butoxycarbonylamino)pentanamido)propanoate

The product of Example 13 (200 mg, 0.317 mmol), N-(3-Dimethylaminopropyl)-N-ethylcarbodiimide hydrochloride (121.5 mg, 0.634 mmol, 2.0 equiv), Boc-L-Lys(Boc)-OH (219.6 mg, 0.634 mmol, 2.0 equiv.), N,N-Diisopropylethylamine (55.2 μL, 0.317 mmol, 1.0 equiv) and 4-Dimethylaminopyridine (catalytic amount) were dissolved in 20 mL of CH₂Cl₂ and stirred at room temperature for sixteen hours. The solvent was removed in vacuo and the resultant residue purified by column chromatography (EtOAc/hexanes) to yield 3-(2,5-Bis-tert-butoxycarbonylamino-pentanoylamino)-propionic acid 4-[2-carbamoyl-5-(6,6-dimethyl-4-oxo-3-trifluoromethyl-4,5,6,7-tetrahydro-indazol-1-yl)-phenylamino]-cyclohexyl ester (61.1 mg, 22.3% yield).

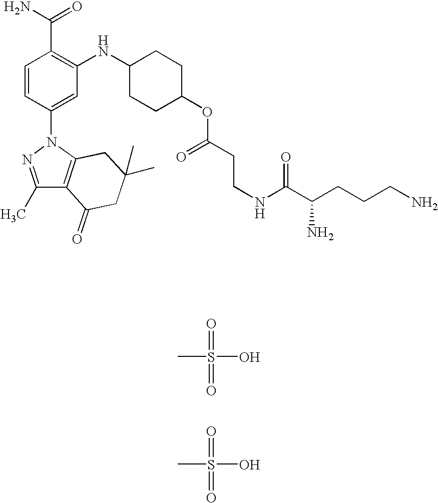

### Example 15

### (S)-4-(2-carbamoyl-5-(6,6-dimethyl-4-oxo-3-(trifluoromethyl)-4,5,6,7-tetrahydro-1H-indazol-1-yl)phenylamino)cyclohexyl 3-(2,5-diaminopentanamido)propanoate dimethanesulfonate

The product of Example 14 was dissolved in 3 mL of CH₂Cl₂ and cooled to 0° C. in an ice-bath. Three milliliters of trifluoroacetic acid was added and the reaction mixture stirred at room temperature for two hours. The solvent was removed in vacuo, and the resultant residue diluted with water. After neutralizing the solution by addition of saturated aqueous sodium bicarbonate, the aqueous suspension was extracted with EtOAc ×3. The combined organics were washed with brine ×2, dried over sodium sulfate, and concentrated in vacuo to yield 3-(2,6-Diamino-hexanoylamino)-propionic acid 4-[2-carbamoyl-5-(6,6-dimethyl-4-oxo-3-trifluoromethyl-4,5,6,7-tetrahydro-indazol-1-yl)-phenylamino]-cyclohexyl ester as an off-white solid (22.3 mg, 10.6% yield). The methane sulfonate was formed by dissolving in 1:1 CH₂Cl₂:MeOH, adding two equivalents of CH₃SO₃H, stirring for one hour, and concentrating in vacuo. LCMS M+1=664.4

Examples 16-41, shown below in Table 1, can be prepared essentially according to the synthetic methodology described herein, Examples 1-15 and Schemes 1-5, and/or by using methodology well known in the art.

TABLE 1

| | | |
|---|---|---|
| Example 16 | 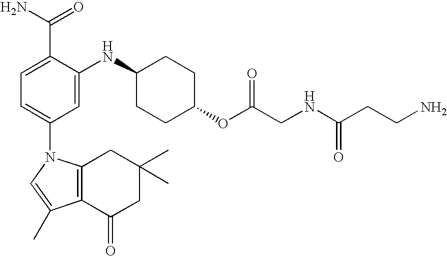 | trans-4-(2-carbamoyl-5- (3,6,6-trimethyl-4-oxo- 4,5,6,7-tetrahydro-1H- indol-1- yl)phenylamino)cyclohexyl 2-(3- aminopropanamido)acetate |
| Example 17 | 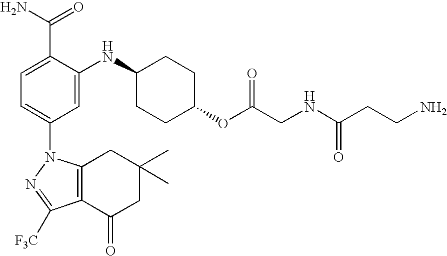 | trans-4-(2-carbamoyl-5- (6,6-dimethyl-4-oxo-3- (trifluoromethyl)- 4,5,6,7-tetrahydro-1H- indazol-1- yl)phenlyamino)cyclohexyl 2-(3- aminopropanamido)acetate |
| Example 18 | 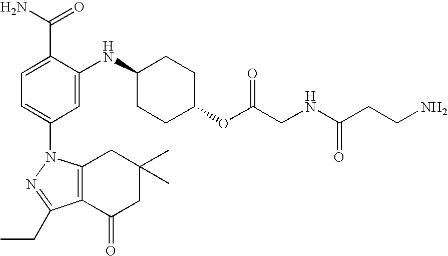 | trans-4-(2-carbamoyl-5- (3-ethyl-6,6-dimethyl-4- oxo-4,5,6,7-tetrahydro- 1H-indazol-1- yl)phenylamnio)cyclohexyl 2-(3- aminopropanamido)acetate |
| Example 19 | 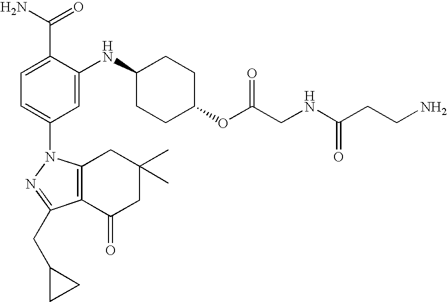 | trans-4-(2-carbamoyl-5- (3-(cylcopropylmethyl)- 6,6-dimethyl-4-oxo- 4,5,6,7-tetrahydro-1H- indazol-1- yl)phenylamino)cyclohexyl 2-(3- 1 2-(3- amniopropanamido)acetate |
| Example 20 | 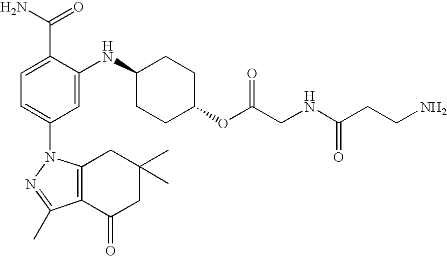 | trans-4-(2-carbamoyl-5- (3,6,6-trimethyl-4-oxo- 4,5,6,7-tetrahydro-1H- indazol-1- yl)phenylamino)cylcohexyl 2-(3- aminopropanamido)acetate |
| Example 21 | 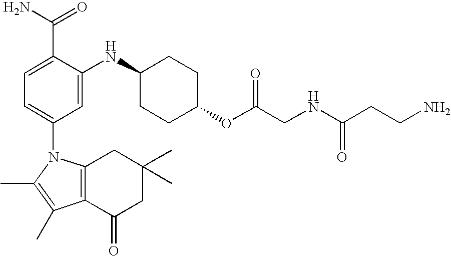 | trans-4-(2-carbamoyl-5- (2,3,6,6-tetramethyl-4- oxo-4,5,6,7-tetrahydro 1H-indol-1- yl)phenylamino)cyclohexyl 2-(3- aminopropanamido)acetate |
| Example 22 | 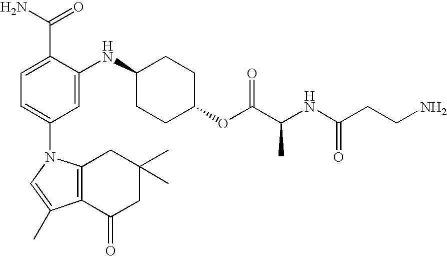 | (S)-(trans-4-(2- carbamoyl-5-(3,6,6- trimethyl-4-oxo-4,5,6,7 tetrahydro-1H-indol-1- yl)phenylamino)cylcohexyl) 2-(3- aminopropanamido)propano- ate |
| Example 23 | 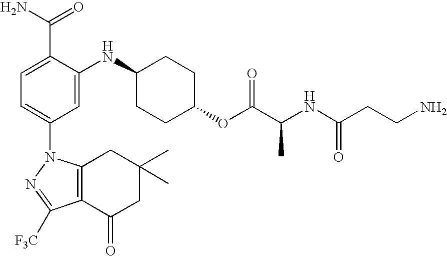 | (S)-(trans-4-(2- carbamoyl-5-(6,6- dimethyl-4-oxo-3- (trifluoromethyl)- 4,5,6,7-tetrahydro-1H- indazol-1- yl)phenylamino)cyclohexyl) 2-(3- aminopropanamido)propano- ate |
| Example 24 | 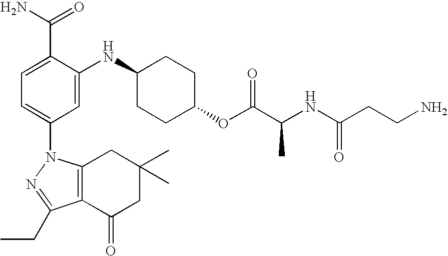 | (S)-(trans-4-(2- carbamoyl-5-(3-ethyl 6,6-dimethyl-4-oxo- 4,5,6,7-tetrahydro-1H- indazol-1- yl)phenylamino)cyclohexyl) 2-(3- aminopropanamido)propano- ate |
| Example 25 | 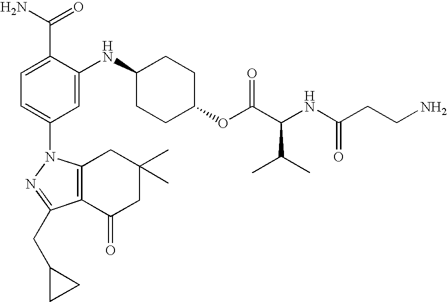 | (S)-(trans-4-(2- carbamoyl-5-(3- (cyclopropylmethyl)-6,6- dimethyl-4-oxo-4,5,6,7- tetrahydro-1H-indazol-1- yl)phenylamino)cyclohexyl) 2-(3- aminopropanamido)-3- methylbutanoate |
| Example 26 | 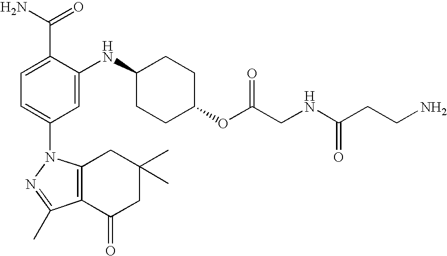 | trans-4-(2-carbamoyl-5- (3,6,6-trimethyl-4-oxo- 4,5,6,7-tetrahydro-1H- indazol-1- yl)phenylamino)cycohexyl 2-(3- aminopropanamido)acetate |
| Example 27 | 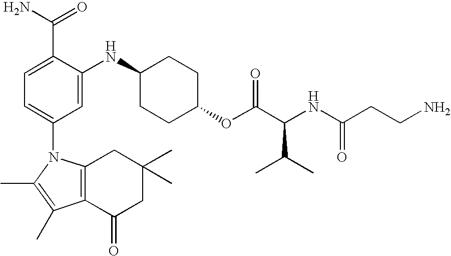 | (S)-(trans-4-(2- carbamoyl-5-(2,3,6,6- tetramethyl-4-oxo- 4,5,6,7-tetrahydro-1H- indo-1- yl)phenylamino)cyclohexyl) 2-(3- aminopropanamido)-3- methylbutanoate |
| Example 28 | 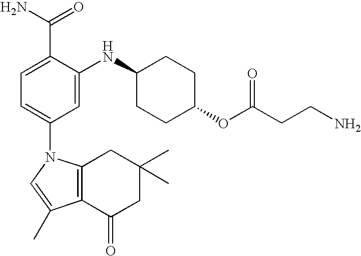 | trans-4-(2-carbamoyl-5- (3,6,6-trimethyl-4-oxo- 4,5,6,7-tetrahydro-1H- indo-1- yl)phenylamino)cyclohexyl 3-aminopropanoate |
| Example 29 | 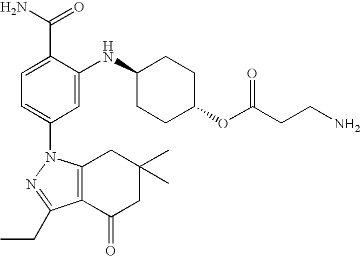 | trans-4-(2-carbamoyl-5- (3-ethyl-6,6-dimethyl-4- oxo-4,5,6,7-tetrahydro- 1H-indazol-1- yl)phenylamino)cyclohexyl 3-aminopropanoate |
| Example 30 | 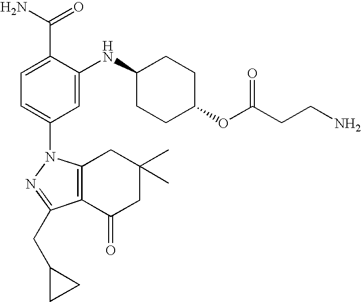 | trans-4-(2-carbamoyl-5- (3-(cyclopropylmethyl)- 6,6-dimethyl-4-oxo- 4,5,6,7-tetrahydro-1H- indazol-1- yl)phenylamino)cyclohexyl 3-aminopropanoate |
| Example 31 | 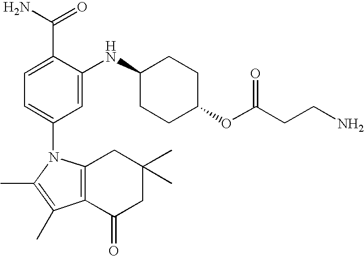 | trans-4-(2-carbamoyl-5- (2,3,6,6-tetramethyl-4- oxo-4,5,6,7-tetrahydro- 1H-indol-1- yl)phenylamino)cyclohexyl 3-aminopropanoate |
| Example 32 | 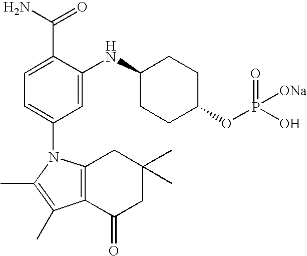 | sodium trans-4-(2- carbamoyl-5-(2,3,6,6- tetramethyl-4-oxo- 4,5,6,7-tetrahydro-1H- indol-1- yl)phenylamino)cyclohexyl hydrogenphosphate |
| Example 33 | 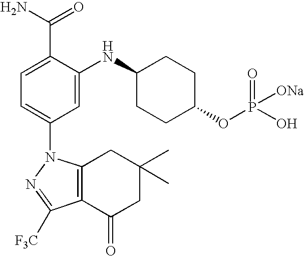 | sodium trans-4-(2- carbamoyl-5-(6,6- dimethyl-4-oxo-3- (trifluoromethyl)- 4,5,6,7-tetrahydro-1H- indazol-1- yl)phenylamino)cyclohexyl hydrogenphosphate |
| Example 34 | 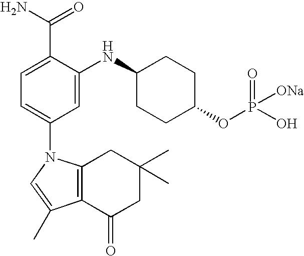 | sodium trans-4-(2- carbamoyl-5-(3,6,6- trimethyl-4-oxo-4,5,6,7- tetrahydro-1H-indol-1- yl)phenylamino)cyclohexyl hydrogenphosphate |
| Example 35 | 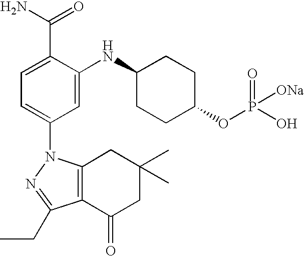 | sodium trans-4-(2- carbamoyl-5-(3-ethyl- 6,6-dimethyl-4-oxo- 4,5,6,7-tetrahydro-1H- indazol-1- yl)phenylamino)cyclohexyl hydrogenphosphate |
| Example 36 | 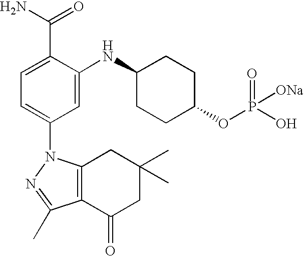 | sodium trans-4-(2- carbamoyl-5-(3,6,6- trimethyl-4-oxo-4,5,6,7- tetrahydro-1H-indazol-1- yl)phenylamino)cyclohexyl hydrogenphosphate |
| Example 37 | 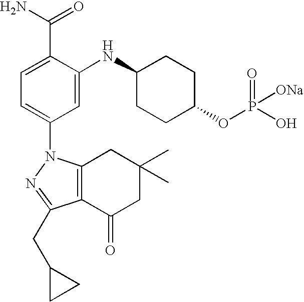 | sodium trans-4-(2- carbamoyl-5-(3- (cyclopropylmethyl)-6,6- dimethyl-4-oxo-4,5,6,7- tetrahydro-1H-indazol-1- yl)phenylamino)cyclohexyl hydrogenphosphate |
| Example 38 | 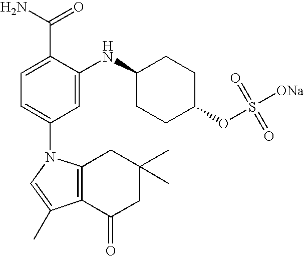 | sodium trans-4-(2- carbamoyl-5-(3,6,6- trimethyl-4-oxo-4,5,6,7- tetrahydro-1H-indol-1- yl)phenylamino)cyclohexyl sulfate |
| Example 39 | 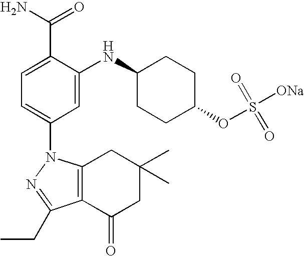 | sodium trans-4-(2- carbamoyl-5-(3-ethyl- 6,6-dimethyl-4-oxo- 4,5,6,7-tetrahydro-1H- indazol-1- yl)phenylamino)cyclohexyl sulfate |
| Example 40 | 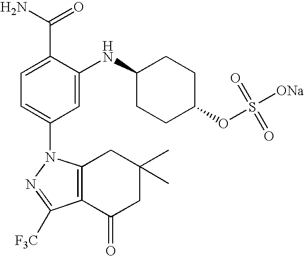 | sodium trans-4-(2- carbamoyl-5-(6,6- dimethyl-4-oxo-3- (trifluoromethyl)- 4,5,6,7-tetrahydro-1H- indazol-1- yl)phenylamino)cyclohexyl sulfate |
| Example 41 | 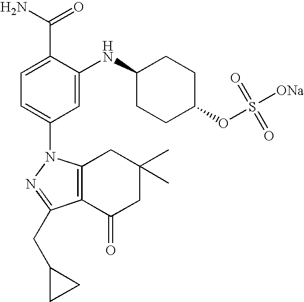 | sodium trans-4-(2- carbamoyl-5-(3- (cyclopropylmethyl)-6,6- dimethyl-4-oxo-4,5,6,7- tetrahydro-1H-indazol-1- yl)phenylamino)cyclohexyl sulfate |

Biological Evaluation

### Example 42

### Cell Proliferation Assays

A panel of cancer cell lines was obtained from the DCTP Tumor Repository, National Cancer Institute (Frederick, Md.) or ATCC (Rockville, Md.). Cell cultures were maintained in Hyclone RPMI 1640 medium (Logan, Utah) supplemented with 10% fetal bovine serum and 20 mM HEPES buffer, final pH 7.2, at 37° C. with a 5% CO₂ atmosphere. Cultures were maintained at sub-confluent densities. Human umbilical vein endothelial cells (HUVEC) were purchased from Clonetics, a division of Cambrex (Walkersville, Md.). Cultures were established from cryopreserved stocks using Clonetics EGM-2 medium supplemented with 20 mM HEPES, final pH 7.2, at 37° C. with a 5% CO₂ atmosphere.

For proliferation assays, cells were seeded with the appropriate medium into 96 well plates at 1,000-2,500 cells per well, depending on the cell line, and were incubated overnight. The following day, test compound, DMSO solution (negative control), or Actinomycin D (positive control) was added to the appropriate wells as 10× concentrated stocks prepared in phosphate buffered saline. The cell plates were then incubated for an additional 2-5 days, depending on the cell line, to allow proliferation to occur. To measure cell density, 50 μL of WST-1 solution (Roche Applied Science, IN) diluted 1:5 in phosphate buffered saline was added to each well, and the cells incubated for an additional 1-5 hrs., again depending on the cell line. Optical density was determined for each well at 450 nM using a Tecan GeniosPro plate reader (RTP, NC). The percentage of cell growth was determined by comparing the cell growth in the presence of test compounds to the cells treated with DMSO vehicle (control, 100% growth) and cells treated with Actinomycin D (10 μM, 0% growth).

Immediately after the WST-1 determination, the medium was removed from the PC-3, NCI-H460 and HUVEC cell lines, and the plates stored at −80° C. Using these assay plates, relative amounts of DNA in each well were determined using the Cyquant DNA assay kit from R&D Systems (Eugene, Oreg.) following the manufacturer's directions. Results for each compound treatment were compared to DMSO vehicle control (100%) and 10 μM Actinomycin D treated cells (0%).

Several exemplary compounds useful in the methods of the invention are listed below. The range of their inhibitory activity against PC-3 cell proliferation is demonstrated, where A stands for an IC₅₀ value that is less than 0.5 μM, B between 0.5 and 5 μM, C between 5 and 50 μM.

| | |
|---|---|
| 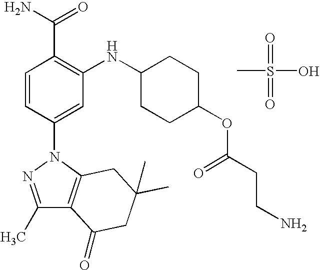 | B |
| 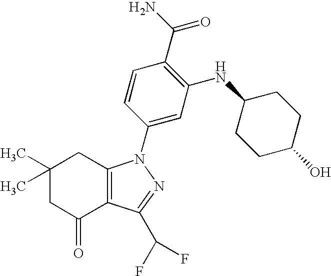 | A |
| 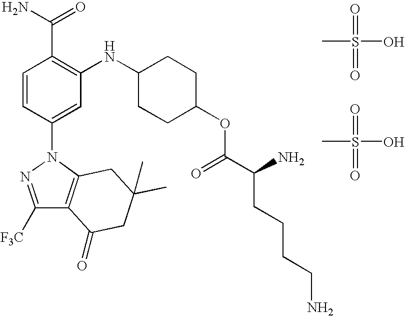 | A |
| 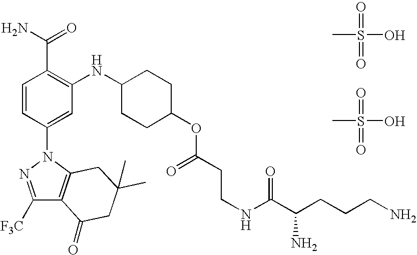 | A |
| 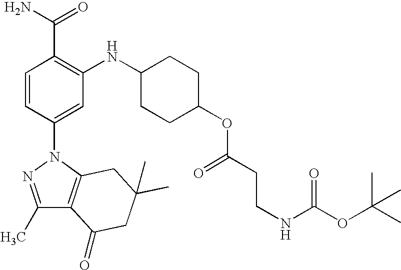 | A |
| 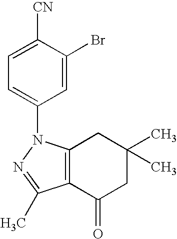 | C |

### Example 43

### Determination of Affinity for HSP-90

(Heat Shock Protein 90)

Affinity of test compounds for HSP-90 was determined as follows: Protein mixtures obtained from a variety of organ tissues (for example: spleen, liver and lung) were reversibly bound to a purine affinity column to capture purine-binding proteins, especially HSP-90. The purine affinity column was washed several times, and then eluted with 20μM, 100 μM, and 500 μM of test compound. Compounds of Formula I elute HP-90 in a dose-dependent manner vs. a control elution using dimethylsulfoxide. The elution profile of Formula I compounds was determined by 1-dimensional SDS polyacrylamide gel electrophoresis. Gels were stained with a fluorescent stain such as sypro ruby (a highly sensitive fluorescent protein stain that can readily detect less than 1 fmol of total protein, i.e., less than 0.04 ng for a 40 kDa protein) or silver nitrate. The gels were imaged using a standard flat bed gel imager and the amount of protein estimated by densitometry. The percent of HSP-90 protein eluted from the column at each concentration was determined and IC₅₀ values were calculated from these estimates. The identity of a band containing HSP-90 was determined by protein sequencing using mass spectroscopy.

Compounds of the invention are inhibitors of HSP-90 (heat shock protein 90). Several exemplary compounds useful in the methods of the invention are listed below. The range of their relative binding affinity to HSP-90 is demonstrated, where A stands for very high, B for high and C for moderate.

| | |
|---|---|
| 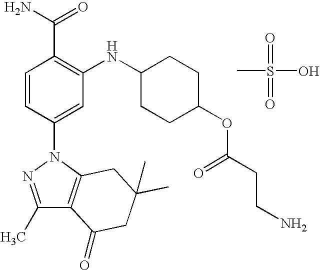 | A |
| 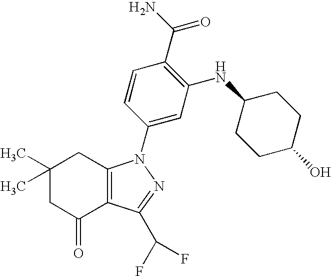 | A |
| 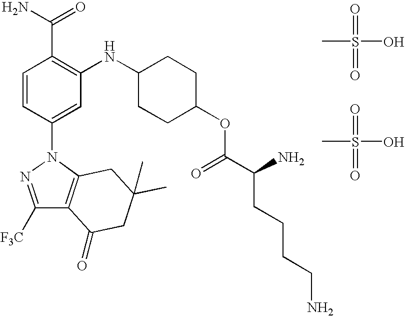 | A |
| 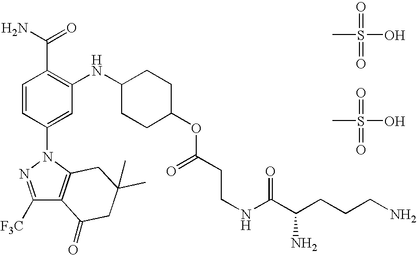 | A |
| 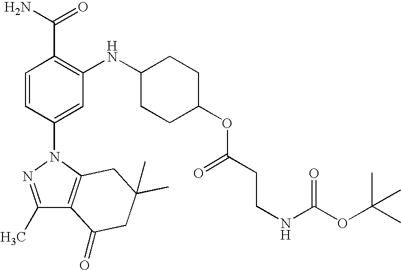 | A |
| 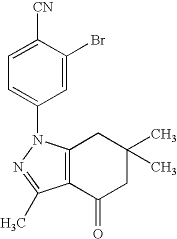 | C |

The invention and the manner and process of making and using it, are now described in such full, clear, concise and exact terms as to enable any person skilled in the art to which it pertains, to make and use the same. It is to be understood that the foregoing describes preferred embodiments of the invention and that modifications may be made therein without departing from the spirit or scope of the invention as set forth in the claims. To particularly point out and distinctly claim the subject matter regarded as invention, the following claims conclude this specification.

What is claimed is:

1. A compound of the Formula (I) 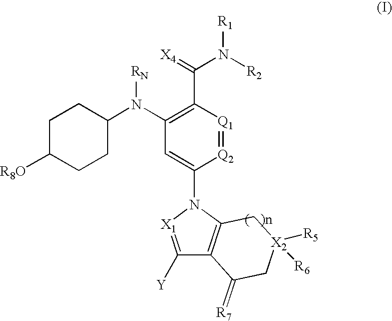
or a pharmaceutically acceptable salt thereof, wherein
Q₁ and Q₂ are independently N or CR_{Q}, wherein
each R_{Q} is independently hydrogen, halogen, cyano, —C(O)OH, —C(O)—O(C₁-C₆ alkyl), —N(R_{N})₂, C₁-C₆ alkyl, C₁-C₆ haloalkyl, C₃-C₇ cycloalkyl, aryl, or heteroaryl, wherein
each alkyl, cycloalkyl, aryl, and heteroaryl group is optionally substituted with from 1-4 groups that are independently C₁-C₆ alkyl, C₁-C₆ alkoxy, halogen, hydroxy, amino, mono- or di-(C₁-C₆)alkylamino, halo(C₁-C₆)alkyl, halo(C₁-C₆)alkoxy, or carboxamide;
each R_{N} is independently —H, —C₁-C₆ alkyl-, —C₁-C₆ haloalkyl, or —C(O)Z, wherein
Z is —C₁-C₆ alkyl, —C₁-C₆ haloalkyl, -aryl, —OR′, wherein R′ is —C₁-C₆ alkyl, —C₁-C₆ haloalkyl, cycloalkyl, aryl, heterocycloalkyl, or heteroaryl; wherein
each alkyl, cycloalkyl, aryl, heterocycloalkyl, and heteroaryl group is optionally substituted with from 1-4 groups that are independently C₁-C₆ alkyl, C₁-C₆ alkoxy, halogen, hydroxy, amino, mono- or di-(C₁-C₆)alkylamino, halo(C₁-C₆)alkyl, halo(C₁-C₆)alkoxy, or carboxamide;
R₁ and R₂ are independently H or C₁-C₆ alkyl;
R₇ is O, S, NH, N—OH, N—NH₂, N—NHR′, or N—O—R;
R₈ is —P(O)(OR_{P})₂, —S(O)₂OR_{S}, or -T-V—NH(R₂₀);
each R_{P} is —H, —C₁-C₁₀ alkyl, —C₁-C₁₀ haloalkyl, or aryl, wherein
the C₁-C₁₀ alkyl or aryl is optionally substituted with one
or more groups independently selected from C₁-C₆ alkyl, C₁-C₆ alkoxy, halogen, hydroxy, amino, mono- or di-(C₁-C₆)alkylamino, nitro, halo(C₁-C₆)alkyl, carboxy, or carboxamide;
R_{S} is —H, —C₁-C₁₀ alkyl, -C₁-C₁₀ haloalkyl, or aryl, wherein the C₁-C₁₀ alkyl or aryl is optionally substituted with one or more groups indpendently selected from C₁-C₆ alkyl, C₁-C₆ alkoxy, halogen, cyano, hydroxy, amino, mono- or di-(C₁-C₆)alkylamino, nitro, halo(C₁-C₆)alkyl, carboxy, or carboxamide;
T is —C(O)—, —C(O)O—, —C(O)N(R_{N})—, —S(O)—, or —S(O)₂;
V is C₂-C₁o alkyl optionally substituted with at least one group
selected from C₁-C₆ alkyl, C₁-C₆ alkoxy, halogen, hydroxy, amino, mono- or di-(C₁-C₆)alkylamino, nitro, halo(C₁-C₆)alkyl, carboxy, carboxamide, or —NH—C(O)—C₁-C₆ alkoxy; or
V is V₂-L-V₁;
V₁ is —C₁-C₁₀ alkyl-, —C₃-C₇ cycloalkyl-, -aryl-, or -heteroaryl-;
L is —O—, —C(O)—, —OC(O), —C(O)O—, —N(R_{N})C(O)—, —C(O)N(R_{N})—, —OC(O)N(R_{N})—, —N(R_{N})C(O)O—, or —N(R_{N})C(O)N(R_{N})—;
V₂ is —C₁-C₁₀ alkyl-, —C₃-C₇ cycloalkyl-, -aryl-, or -heteroaryl-;
wherein each V₁ and V₂ independently are optionally substituted with at least one group selected from C₁-C₆ alkyl, C₁-C₆ alkoxy, halogen, hydroxy, amino, mono- or
di-(C₁-C₆)alkylamino, nitro, halo(C₁-C₆)alkyl, carboxy, carboxamide, or —NH—C(O)—C₁-C₆ alkoxy;
R₂₀ is —H or —C(O)—C₁-C₆ alkoxy;
X₁ represents N or CR_{X} wherein R_{X} is —H or C₁-C₆ alkyl;
X₂ is C or N;
X₄ is O, S, NH, N—OH, N—NH₂, N—NHaryl, N—NH—(C₁-C₆ alkyl), or N—O—R′;
Y is hydrogen, halogen, C₁-C₁₀ alkyl, C₁-C₁₀ haloalkyl,
C₃-C₇ cycloalkyl, C₃-C₇ cycloalkyl(C₁-C₁₀)alkyl, C₁-C₆ acyl, aryl or heteroaryl, wherein
each C₁-C₁₀ alkyl or C₃-C₇ cycloalkyl group is optionally substituted with aryl or heteroaryl, wherein
the aryl and heteroaryl groups are optionally substituted with 1-4 groups that are independently C₁-C₆ alkyl, C₁-C₆ alkoxy, halogen, hydroxy, amino, mono- or di-(C₁-C₆)alkylamino, nitro, halo(C₁-C₆)alkyl, halo(C₁-C₆)alkoxy, or carboxamide; and
n is 0, 1, 2, 3, or 4,
provided that when
(i) X₂ is C, then
R₅ and R₆ are independently H or C₁-C₆ alkyl, or
R₅ and R₆ together with the carbon to which they are attached form a 5-8 membered ring; and
(ii) X₂ is N, then
R₆ is absent and R₅ is H or C₁-C₆ alkyl.

2. A compound according to claim 1 of Formula (II) 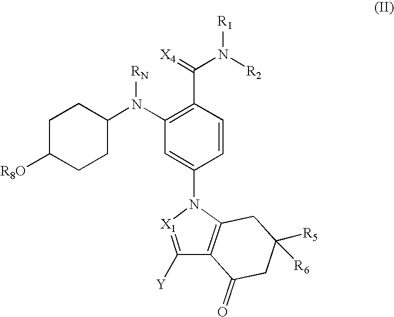
or a pharmaceutically acceptable salt thereof.

3. A compound according to claim 2 wherein
R₅ and R₆ are independently H or C₁-C₆ alkyl.

4. A compound according to claim 3, wherein
R₅ and R₆ are independently H or C₁-C₆ alkyl; and
X₄ is O.

5. A compound according to claim 1 of Formula (III) 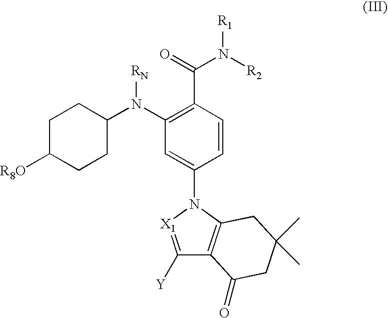
or a pharmaceutically acceptable salt thereof.

6. The compound according to claim 5, wherein R_{N} is —H or —C₁-C₁₀ alkyl.

7. A compound according to claim 5, wherein X₁ is N CH or C(CH₃).

8. A compound according to claim 1 of Formula (IV) 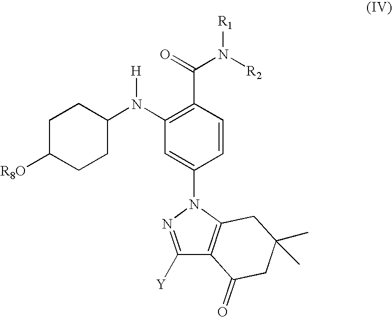
or a pharmaceutically acceptable salt thereof.

9. A compound according to claim 8, wherein Y is hydrogen, halogen, C₁-C₁₀ alkyl, C₃-C₇ cycloalkyl(C₁-C₁₀)alkyl, or C₁-C₁₀ haloalkyl.

10. A compound according to claim 8, wherein
R₈ is -T-V—NH(R₂₀);
T is —C(O)—, —C(O)O—, —C(O)N(R_{N})—, —S(O)—, or —S(O)₂;
V is C₂-C₁₀ alkyl optionally substituted with at least one group selected from C₁-C₆ alkyl, C₁-C₆ alkoxy, halogen, hydroxy, amino, mono- or di-(C₁-C₆)alkylamino, nitro, halo(C₁-C₆)alkyl, carboxy, carboxamide, or —NH—C(O)—C₁-C₆ alkoxy; or
V is V₂-L-V₁;
V₁ is —C₁-C₁₀ alkyl-, —C₃-C₇ cycloalkyl-, -aryl-, or -heteroaryl-;
L is —O—, —C(O)—, —OC(O), —C(O)O—, —N(R_{N})C(O)—, —C(O)N(R_{N})—, —OC(O)N(R_{N})—, —N(R_{N})C(O)O—, or —N(R_{N})C(O)N(R_{N})—; and
V₂ is —C₁-C₁₀ alkyl-, —C₃-C₇ cycloalkyl-, -aryl-, or -heteroaryl-; wherein each V₁ and V₂ independently is optionally substituted with at least one group which is
C₁-C₆ alkyl, C₁-C₆ alkoxy, halogen, hydroxy, amino, mono- or di-(C₁-C₆)alkylamino, nitro, halo(C₁-C₆)alkyl, carboxy, or carboxamide; R₂₀ is —H or —C(O)—C₁-C₆ alkoxy;

11. A compound according to claim 1 of Formula (V) 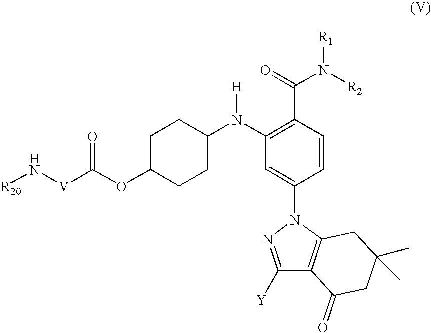
or a pharmaceutically acceptable salt thereof.5

12. A compound according to claim 11, wherein
Y is hydrogen, halogen, C₁-C₁₀ alkyl, C₃-C₇ cycloalkyl(C₁-C₁₀)alkyl, or C₁-C₁₀ haloalkyl;
V is —C₂-C₁₀ alkyl- optionally substituted with at least one group selected from C₁-C₆ alkyl, C₁-C₆ alkoxy, halogen, hydroxy, amino, mono- or di-(C₁-C₆)alkylamino, nitro, halo(C₁-C₆)alkyl, carboxy, carboxamide, or —NH—C(O)—C₁-C₆ alkoxy; or
V is V₂-L-V₁;
V₁ is —C₂-C₁₀ alkyl- optionally substituted with at least one group selected from C₁-C₆ alkyl, C₁-C₆ alkoxy, halogen, hydroxy, amino, mono- or di-(C₁-C₆)alkylamino, nitro, halo(C₁-C₆)alkyl, carboxy, carboxamide, or —NH—C(O)—C₁-C₆ alkoxy;
L is —N(R_{N})C(O)—; and
V₂ is —C₂-C₁₀ alkyl- optionally substituted with at least one group selected from C₁-C₆ alkyl, C₁-C₆ alkoxy, halogen, hydroxy, amino, mono- or di-(C₁-C₆)alkylamino, nitro, halo(C₁-C₆)alkyl, carboxy, carboxamide, or —NH—C(O)—C₁-C₆ alkoxy.

13. A compound according to claim 1 of Formula (VI) 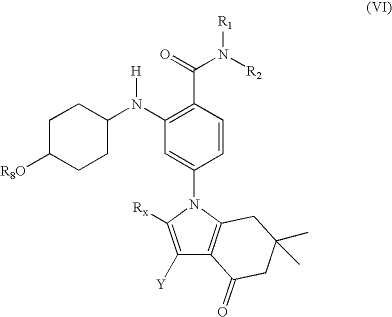
or a pharmaceutically acceptable salt thereof.

14. A compound according to claim 13, wherein
Y is hydrogen, halogen, C₁-C₁₀ alkyl, C₃-C₇ cycloalkyl(C₁-C₁₀)alkyl, or C₁-C₁₀ haloalkyl; and
R_{X} is H or CH.

15. A compound according to claim 13, wherein
Y is hydrogen, halogen, C₁-C₁₀ alkyl, C₃-C₇ cycloalkyl(C₁-C₁₀)alkyl, or C₁-C₁₀ haloalkyl;
R₈ is -T-V—NH(R₂₀);
T is —C(O)—, —C(O)O—, —C(O)N(R_{N})—, —S(O)—, or —S(O)₂;
V is C₂-C₁o alkyl optionally substituted with at least one group selected from C₁-C₆ alkyl, C₁-C₆ alkoxy, halogen, hydroxy, amino, mono- or di-(C₁-C₆)alkylamino, nitro, halo(C₁-C₆)alkyl, carboxy, carboxamide, or —NH—C(O)—C₁-C₆ alkoxy; or
V is V₂-L-V₁;
V₁ is —C₁-C₁₀ alkyl-, —C₃-C₇ cycloalkyl-, -aryl-, or -heteroaryl-;
L is —O—, —C(O)—, —OC(O), —C(O)O—, —N(R_{N})C(O)—, —C(O)N(R_{N})—, —OC(O)N(R_{N})—, —N(R_{N})C(O)O—, or —N(R_{N})C(O)N(R_{N})—; and
V₂ is —C₁-C₁₀ alkyl-, —C₃-C₇ cycloalkyl-, -aryl-, or -heteroaryl-; each V₁ and V₂ independently are optionally substituted with at least one group selected from C₁-C₆ alkyl, C₁-C₆ alkoxy, halogen, hydroxy, amino, mono- or di-(C₁-C₆)alkylamino, nitro, halo(C₁-C₆)alkyl, carboxy, carboxamide, or —NH—C(O)—C₁-C₆ alkoxy.

16. A compound according to claim 1 of Formula (VII) 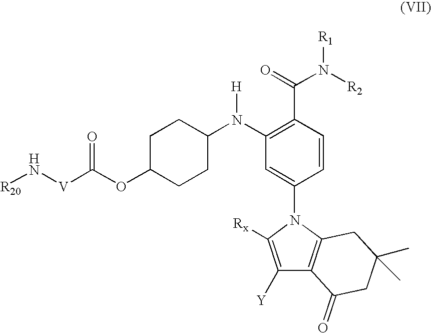
or a pharmaceutically acceptable salt thereof.

17. A compound according to claim 16, wherein
R_{X} is H or CH₃;
Y is hydrogen, halogen, C₁-C₁₀ alkyl, C₃-C₇ cycloalkyl(C₁-C₁₀)alkyl, or C₁-C₁₀ haloalkyl;
V is —C₂-C₁₀ alkyl- optionally substituted with at least one group selected from C₁-C₆ alkyl, C₁-C₆ alkoxy, halogen, hydroxy, amino, mono- or di-(C₁-C₆)alkylamino, nitro, halo(C₁-C₆)alkyl, carboxy, carboxamide, or —NH—C(O)—C₁-C₆ alkoxy; or
V is V₂-L-V₁;
V₁ is —C₂-C₁₀ alkyl- optionally substituted with at least one group selected from C₁-C₆ alkyl, C₁-C₆ alkoxy, halogen, hydroxy, amino, mono- or di-(C₁-C₆)alkylamino, nitro, halo(C₁-C₆)alkyl, carboxy, carboxamide, or —NH—C(O)—C₁-C₆ alkoxy;
L is —O—, —OC(O), —C(O)O—, —N(R_{N})C(O)—, or —C(O)N(R_{N})—; and
V₂ is —C₂-C₁₀ alkyl- optionally substituted with at least one group selected from C₁-C₆ alkyl, C₁-C₆ alkoxy, halogen, hydroxy, amino, mono- or di-(C₁-C₆)alkylamino, nitro, halo(C₁-C₆)alkyl, carboxy, carboxamide, or —NH—C(O)—C₁-C₆ alkoxy.

18. A compound according to claim 16, wherein
R_{X} is H or CH₃;
Y is hydrogen, halogen, C₁-C₁₀ alkyl, C₃-C₇ cycloalkyl(C₁-C₁₀)alkyl, or C₁-C₁₀ haloalkyl;
V is —C₂-C₁₀ alkyl- optionally substituted with at least one group selected from C₁-C₆ alkyl, C₁-C₆ alkoxy, halogen, hydroxy, amino, mono- or di-(C₁-C₆)alkylamino, nitro, halo(C₁-C₆)alkyl, carboxy, carboxamide, or —NH—C(O)—C₁-C₆ alkoxy; or
V is V₂-L-V₁;
V₁ is —C₂-C₁₀ alkyl- optionally substituted with at least one group selected from C₁-C₆ alkyl, C₁-C₆ alkoxy, halogen, hydroxy, amino, mono- or di-(C₁-C₆)alkylamino, nitro, halo(C₁-C₆)alkyl, carboxy, carboxamide, or —NH—C(O)—C₁-C₆ alkoxy;
L is —N(R_{N})C(O)—; and
V₂ is —C₂-C₁₀ alkyl- optionally substituted with at least one group selected from C₁-C₆ alkyl, C₁-C₆ alkoxy, halogen, hydroxy, amino, mono- or di-(C₁-C₆)alkylamino, nitro, halo(C₁-C₆)alkyl, carboxy, carboxamide, or —NH—C(O)—C₁-C₆ alkoxy.

19. A compound according to claim 1 of Formula (VIII) 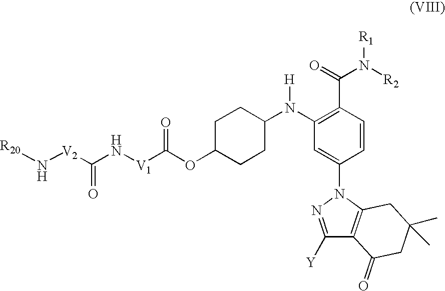
or a pharmaceutically acceptable salt thereof.

20. A compound according to claim 1 of the Formula (IX) 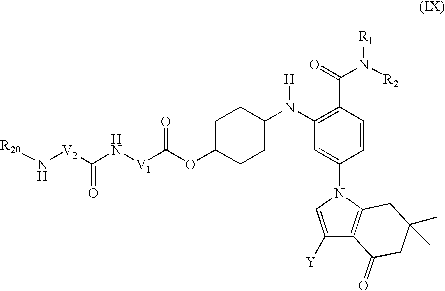
or a pharmaceutically acceptable salt thereof.

21. A compound according to claim 1 of the Formula (X) 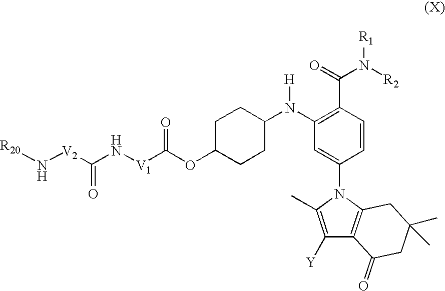
or a pharmaceutically acceptable salt thereof.

22. A compound according to claim 1 selected from the group consisting of
trans-4-(2-carbamoyl-5-(6,6-dimethyl-4-oxo-3-(trifluoromethyl)-4,5,6,7-tetrahydro-1H-indazol-1-yl)phenylamino)cyclohexyl 3-aminopropanoate;
(S)-(trans-4-(2-carbamoyl-5-(6,6-dimethyl-4-oxo-3-(trifluoromethyl)-4,5,6,7-tetrahydro-1H-indazol-1-yl)phenylamino)cyclohexyl) 2,6-bis(tert-butoxycarbonylamino)hexanoate;
(S)-(trans-4-(2-carbamoyl-5-(6,6-dimethyl-4-oxo-3-(trifluoromethyl)-4,5,6,7-tetrahydro-1H-indazol-1-yl)phenylamino)cyclohexyl) 2,6-diaminohexanoate;
4-(2-carbamoyl-5-(3,6,6-trimethyl-4-oxo-4,5,6,7-tetrahydro-1H-indazol-1-yl)phenylamino)cyclohexyl 3-(tert-butoxycarbonylamino)propanoate;
4-(2-carbamoyl-5-(3,6,6-trimethyl-4-oxo-4,5,6,7-tetrahydro-1H-indazol-1-yl)phenylamino)cyclohexyl 3-aminopropanoate;
(S)-4-(2-carbamoyl-5-(6,6-dimethyl-4-oxo-3-(trifluoromethyl)-4,5,6,7-tetrahydro-1H-indazol-1-yl)phenylamino)cyclohexyl 3-(2,5-bis(tert-butoxycarbonylamino)pentanamido)propanoate; and
(S)-4-(2-carbamoyl-5-(6,6-dimethyl-4-oxo-3-(trifluoromethyl)-4,5,6,7-tetrahydro-1H-indazol-1-yl)phenylamino)cyclohexyl 3-(2,5-diaminopentanamido)propanoate;
or a pharmaceutically acceptable salt thereof.

23. A compound according to claim 1 selected from the group consisting of
trans-4-(2-carbamoyl-5-(3,6,6-trimethyl-4-oxo-4,5,6,7-tetrahydro-1H-indol-1-yl)phenylamino)cyclohexyl 2-(3-aminopropanamido)acetate;
trans-4-(2-carbamoyl-5-(6,6-dimethyl-4-oxo-3-(trifluoromethyl)-4,5,6,7-tetrahydro-1H-indazol-1-yl)phenylamino)cyclohexyl 2-(3-aminopropanamido)acetate;
trans-4-(2-carbamoyl-5-(3-ethyl-6,6-dimethyl-4-oxo-4,5,6,7-tetrahydro-1H-indazol-1-yl)phenylamino)cyclohexyl 2-(3-aminopropanamido)acetate;
trans-4-(2-carbamoyl-5-(3-(cyclopropylmethyl)-6,6-dimethyl-4-oxo-4,5,6,7-tetrahydro-1H-indazol-1-yl)phenylamino)cyclohexyl 2-(3-aminopropanamido)acetate;
trans-4-(2-carbamoyl-5-(3,6,6-trimethyl-4-oxo-4,5,6,7-tetrahydro-1H-indazol-1-yl)phenylamino)cyclohexyl 2-(3-aminopropanamido)acetate;
trans-4-(2-carbamoyl-5-(2,3,6,6-tetramethyl-4-oxo-4,5,6,7-tetrahydro-1H-indol-1-yl)phenylamino)cyclohexyl 2-(3-aminopropanamido)acetate;
(S)-(trans-4-(2-carbamoyl-5-(3,6,6-trimethyl-4-oxo-4,5,6,7-tetrahydro-1H-indol-1-yl)phenylamino)cyclohexyl) 2-(3-aminopropanamido)propanoate;
(S)-(trans-4-(2-carbamoyl-5-(6,6-dimethyl-4-oxo-3-(trifluoromethyl)-4,5,6,7-tetrahydro-1H-indazol-1-yl)phenylamino)cyclohexyl) 2-(3-aminopropanamido)propanoate;
(S)-(trans-4-(2-carbamoyl-5-(3-ethyl-6,6-dimethyl-4-oxo-4,5,6,7-tetrahydro-1H-indazol-1-yl)phenylamino)cyclohexyl) 2-(3-aminopropanamido)propanoate;
(S)-(trans-4-(2-carbamoyl-5-(3-(cyclopropylmethyl)-6,6-dimethyl-4-oxo-4,5,6,7-tetrahydro-1H-indazol-1-yl)phenylamino)cyclohexyl) 2-(3-aminopropanamido)-3-methylbutanoate;
trans-4-(2-carbamoyl-5-(3,6,6-trimethyl-4-oxo-4,5,6,7-tetrahydro-1H-indazol-1-yl)phenylamino)cyclohexyl 2-(3-aminopropanamido)acetate;
(S)-(trans-4-(2-carbamoyl-5-(2,3,6,6-tetramethyl-4-oxo-4,5,6,7-tetrahydro-1H-indol-1-yl)phenylamino)cyclohexyl) 2-(3-aminopropanamido)-3-methylbutanoate;
trans-4-(2-carbamoyl-5-(3,6,6-trimethyl-4-oxo-4,5,6,7-tetrahydro-1H-indol-1-yl)phenylamino)cyclohexyl 3-aminopropanoate;
trans-4-(2-carbamoyl-5-(3-ethyl-6,6-dimethyl-4-oxo-4,5,6,7-tetrahydro-1H-indazol-1-yl)phenylamino)cyclohexyl 3-aminopropanoate;
trans-4-(2-carbamoyl-5-(3-(cyclopropylmethyl)-6,6-dimethyl-4-oxo-4,5,6,7-tetrahydro-1H-indazol-1-yl)phenylamino)cyclohexyl 3-aminopropanoate;
trans-4-(2-carbamoyl-5-(2,3,6,6-tetramethyl-4-oxo-4,5,6,7-tetrahydro-1H-indol-1-yl)phenylamino)cyclohexyl 3-aminopropanoate;
sodium trans-4-(2-carbamoyl-5-(2,3,6,6-tetramethyl-4-oxo-4,5,6,7-tetrahydro-1H-indol-1-yl)phenylamino)cyclohexyl hydrogenphosphate;
sodium trans-4-(2-carbamoyl-5-(6,6-dimethyl-4-oxo-3-(trifluoromethyl)-4,5,6,7-tetrahydro-1H-indazol-1-yl)phenylamino)cyclohexyl hydrogenphosphate;
sodium trans-4-(2-carbamoyl-5-(3,6,6-trimethyl-4-oxo-4,5,6,7-tetrahydro-1H-indol-1-yl)phenylamino)cyclohexyl hydrogenphosphate;
sodium trans-4-(2-carbamoyl-5-(3-ethyl-6,6-dimethyl-4-oxo-4,5,6,7-tetrahydro-1H-indazol-1-yl)phenylamino)cyclohexyl hydrogenphosphate;
sodium trans-4-(2-carbamoyl-5-(3,6,6-trimethyl-4-oxo-4,5,6,7-tetrahydro-1H-indazol-1-yl)phenylamino)cyclohexyl hydrogenphosphate;
sodium trans-4-(2-carbamoyl-5-(3-(cyclopropylmethyl)-6,6-dimethyl-4-oxo-4,5,6,7-tetrahydro-1H-indazol-1-yl)phenylamino)cyclohexyl hydrogenphosphate;
sodium trans-4-(2-carbamoyl-5-(3,6,6-trimethyl-4-oxo-4,5,6,7-tetrahydro-1H-indol-1-yl)phenylamino)cyclohexyl sulfate;
sodium trans-4-(2-carbamoyl-5-(3-ethyl-6,6-dimethyl-4-oxo-4,5,6,7-tetrahydro-1H-indazol-1-yl)phenylamino)cyclohexyl sulfate;
sodium trans-4-(2-carbamoyl-5-(6,6-dimethyl-4-oxo-3-(trifluoromethyl)-4,5,6,7-tetrahydro-1H-indazol-1-yl)phenylamino)cyclohexyl sulfate; and
sodium trans-4-(2-carbamoyl-5-(3-(cyclopropylmethyl)-6,6-dimethyl-4-oxo-4,5,6,7-tetrahydro-1H-indazol-1-yl)phenylamino)cyclohexyl sulfate;
or pharmaceutically acceptable salts thereof.

24. A pharmaceutical composition comprising a compound of claim 1, or a pharmaceutically acceptable salt thereof, and a pharmaceutically acceptable solvent, carrier, excipient, adjuvant or a combination thereof.

25. A method of treating cancer, inflammation, or arthritis comprising administration of a therapeutically effective amount of a compound of claim 1, or a pharmaceutically acceptable salt thereof, to a patient in need of such treatment.

26. A package comprising a compound of claim 1 in a container with instructions on how to use the compound.

27. A method of treating a disease or condition related to cell proliferation comprising administration of a therapeutically effective amount of a compound of claim 1, or a pharmaceutically acceptable salt thereof, to a patient in need of such treatment.

28. The method according to claim 27, wherein the disease or condition is cancer, inflammation, or arthritis.

29. A method of treating a disease or disorder related to the activity of heat shock protein 90 comprising administration of a therapeutically effective amount of a compound of claim 1, or a pharmaceutically acceptable salt thereof, to a patient in need of such treatment.

30. The method of claim 29 wherein the disorder is selected from the group consisting of inflammatory diseases, infections, autoimmune disorders, stroke, ischemia, cardiac disorders , neurological disorders, fibrogenetic disorders, proliferative disorders, tumors, leukemias, neoplasms, cancers, carcinomas, metabolic diseases and malignant disease.

31. The method of claim 30 wherein the fibrogenetic disorder is selected from the group consisting of scleroderma, polymyositis, systemic lupus, rheumatoid arthritis, liver cirrhosis, keloid formation, interstitial nephritis and pulmonary fibrosis.

32. A method of treating an infection caused by an organism selected from Plasmodium species comprising administration of a therapeutically effective amount of a compound of claim 1 or a pharmaceutically acceptable salt thereof, to a patient in need of such treatment.

33. A method of reducing the level of infection caused by an organism selected from Plasmodium species comprising administration of a therapeutically effective amount of a compound of claim 1, or a pharmaceutically acceptable salt thereof, to a patient in need of such treatment.

34. A method according to claim 33 where the Plasmodium species is Plasmodium falciparum.

35. A method of treating an infection caused by a a metazoan parasite comprising administration of a therapeutically effective amount of a compound of claim 1, or a pharmaceutically acceptable salt thereof, to a patient in need of such treatment.

36. A method according to claim 35 wherein the metazoan parasite is Plasmodium falciparum.

37. A method for preparing a compound of formula 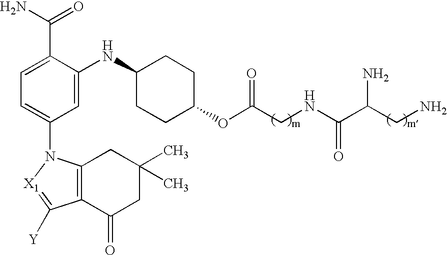
or a pharmaceutically acceptable salt thereof, wherein m is 1-4; m′ is 1-8; X₁ is N or CH; and Y is hydrogen, halogen, C₁-C₁₀ alkyl, C₁-C₁₀ haloalkyl, C₃-C₇ cycloalkyl, and C₃-C₇ cycloalkyl(C₁-Cl₀)alkyl; comprising
(1) treating a compound of formula 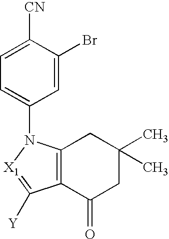
with trans-4-aminocyclohexanol Pd(OAc)₂, DPPF, and a base followed by treatment with EtOH-DMSO and NaOH—H₂O₂ to provide a compound of formula 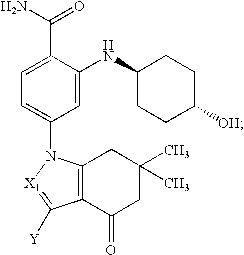
(2) treating the product of (1) with a carbodiimide, DMAP and a compound of formula 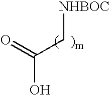
to provide a compound of formula 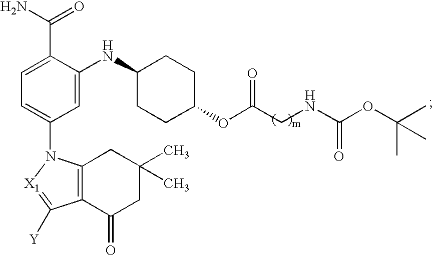
(3) treating the product of (2) with an acid to provide a compound of formula 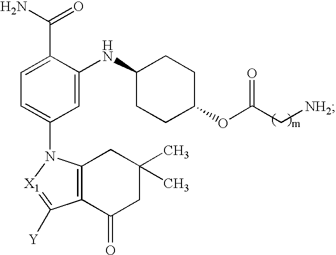
(4) treating the product of (3) with a carbodiimide, DMAP and a compound of formula 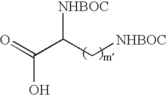
to provide a compound of formula 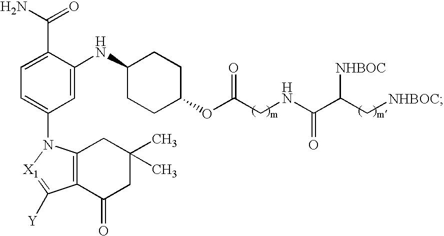
(5) treating the product of (4) with an acid to provide a compound of formula

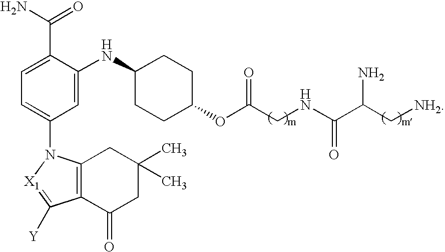

38. A method of preparing a compound of formula 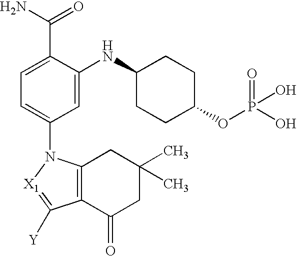
or a pharmaceutically acceptable salt thereof, wherein X₁ is N or CH; and Y is hydrogen, halogen, C₁-C₁₀ alkyl, C₁-C₁₀ haloalkyl, C₃-C₇ cycloalkyl, and C₃-C₇ cycloalkyl(C₁-C₁₀)alkyl; comprising
(1) treating a compound of formula 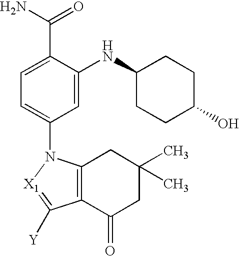
with diphenyl phosphorochloridate, followed by addition
of hydrogen gas, PtO₂, and an acid to provide a compound of formula

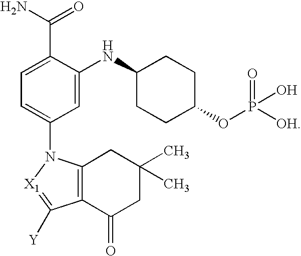

39. A method of preparing a compound of formula 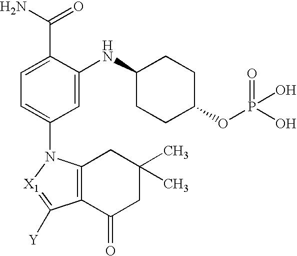
or a pharmaceutically acceptable salt thereof, wherein X₁ is N or CH; and Y is hydrogen, halogen, C₁-C₁₀ alkyl, C₁-C₁₀ haloalkyl, C₃-C₇ cycloalkyl, and C₃-C₇ cycloalkyl(C₁-C₁₀)alkyl; comprising
(1) treating a compound of formula 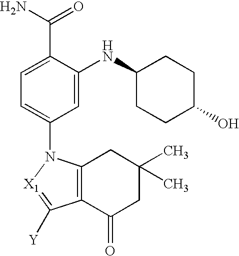
with dibenzyl phosphorochloridate, followed by addition
of hydrogen gas and Pd/C to provide a compound of formula

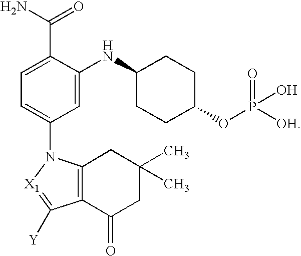

40. A method of preparing a compound of formula 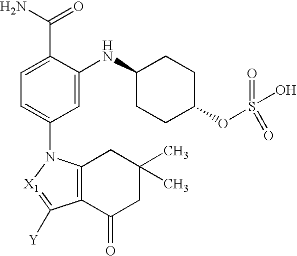
or a pharmaceutically acceptable salt thereof, wherein X₁ is N or CH; and Y is hydrogen, halogen, C₁-C₁₀ alkyl, C₁-C₁₀ haloalkyl, C₃-C₇ cycloalkyl, and C₃-C₇ cycloalkyl(C₁-C₁₀)alkyl; comprising
treating a compound of formula 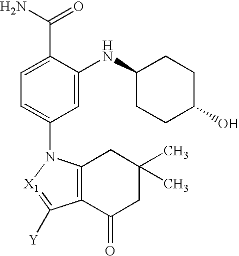
with SO₃ pyridine complex to provide a compound of formula [chemical structure]
